(19) United States
(12) Patent Application Publication
(10) Pub. No.: US 2005/0074425 A1
(43) Pub. Date: Apr. 7, 2005

(54) Method for delivering polymerized therapeutic agent compositions and compositions thereof

(75) Inventors: Jacob Waugh, Palo Alto, CA (US); Mahmood Razavi, Menlo Park, CA (US); Ceron Rhee, Saratoga, CA (US); Clifford Bryant, Millbrae, CA (US)

Correspondence Address: MANATT PHELPS AND PHILLIPS;ROBERT D. BECKER, 1001 PAGE MILL ROAD, BUILDING 2, PALO ALTO, CA, 94304, US

(73) Assignee: Polycord, Inc., Stanford, CA (US)

(21) Appl. No.: 10/884,226

(22) Filed: Jul. 2, 2004

Related U.S. Application Data
(60) Provisional application No. 60/485,076, filed on Jul. 2, 2003.

(51) Int. Cl. A61K 38/17; A61K 31/785; C08G 63/48

(52) U.S. Cl. 424/78.27; 525/54.1

(57) ABSTRACT

A method for delivering polymerized therapeutic agents and their compositions are disclosed. The various polymers take advantage of the functional domains found in a variety of therapeutic agents. The polymerized therapeutic agent compositions are prepared by covalently linking the agent to a biocompatible backbone either directly or through backbone conjugates/monomers. The polymerized therapeutic agent compositions of the invention have highly desirable properties, which make them particularly well suited for use in biological and biomedical applications.

### FIELD OF THE INVENTION

The present invention relates to therapeutic agents and more specifically to delivering novel forms of chemically polymerized therapeutic agent compositions with enhanced biological and pharmacological activity.

### BACKGROUND OF THE INVENTION

The use of therapeutic agents for producing a particular physiological response is well known in the medicinal arts. There are a number of limitations to the potential therapeutic benefits derived from the clinical use of therapeutic agents, including the ability of the agent to elicit the desired response in the circulatory system when biological forces within the body are working against it.

In the past, attempts have been made to conjugate biologically active agents to various forms of matrices to provide desirable distribution of the agent in the body and increase an agent's circulatory half-life. Prior attempts include modification of proteins with substantially straight chain polymers such as polyethylene glycol (PEG) or polypropylene glycol (PPG). See, e.g., Davis et al., U.S. Pat. No. 4,179,337. However, none of the disclosed polymers have the desirable structural feature of having multiple functional groups at regular, predetermined intervals that can be utilized for drug attachment or cross-linking reactions.

Further, while the use of a polymer matrix may increase the amount of agent, passive containment of the agent has a number of critical limitations. First, the polymers used in the matrix will inevitably exert some biologic effect. For example, biodegradable polymers, such as PLGA (a co-polymer of glycolic acid and lactic acid), when hydrolyzed in the vascular environment, may cause a strong local reduction in pH (due to the release of acidic monomers) and thus create a deleterious effect on the treatment. Second, the polymers of the matrix can fragment or embolize, adversely affecting the release characteristic of the matrix and thereby having a potentially direct harmful effect on the patient.

For these reasons, improved polymerized compositions for the presentation of therapeutic agents are desirable. In particular, it would be desirable to avoid the use of polymer matrices for passively sequestering the agent. It would be further desirable if the agent itself could be polymerized or linked to a backbone polymer wherein the linkage can be degradable or non-degradable. It would also be desirable if the agent was able to retain function while in its polymerized form or inactive until processed (pro-drug). In the functional polymerized form, it would be desirable if the active was held in a confirmation that was unique from the original non-polymerized form, thereby enhancing its activity. Further, it would also be beneficial if the therapeutic agent could be linked directly to the polymer generating a polymerized compound of three or more therapeutic agents. It would also be desirable if the therapeutic agent could be linked to a polymer conjugate that could then be polymerized into a polymerized compound of two or more therapeutic agents. Finally, it would also be desirable if the therapeutic agent could be polymerized to itself, generating a polymerized compound of two or more agents. The present invention offers unique methods of polymerizing therapeutic agents that accomplishes these goals.

### BRIEF SUMMARY OF THE INVENTION

Novel compositions and methods are provided for delivering a physiologically and biologically active agent-containing composition to a patient, where the the agent is in the form of a polymerized composition. The compositions are in either their biodegradable or non-biodegradable forms. They are selected from the group consisting of a polymer of at least three of the agents; a polymer of at least three of the agents having a polymerizable moiety polymerized to at least one of the agents; a polymer of at least three of the agents having a backbone molecule covalently bound to at least one of the agents; a polymer where the agent is covalently bound to at least one of the agents through linking moieties.

Novel compositions, methods of preparation of such compositions, and the methods of using polymerizable therapeutic agents and polymers thereof are disclosed. Specifically included are therapeutic agents that can be linked directly via other links to an amino acid. The polymers can be homopolymers or heteropolymers.

### BRIEF DESCRIPTION OF THE DRAWINGS

FIG. 1. Structure of Eptifibatide (INTEGRILIN®);

FIG. 2. Reaction scheme for converting carboxylates to amine-reactive structures for amide-based crosslinking;

FIG. 3. Reaction scheme for converting carboxylates to hydroxyl-reactive structures for amide-based crosslinking;

FIG. 4. Structure of ascorbic acid;

FIG. 5. Structure of losartan;

FIG. 6. Structure of atorvastatin;

FIG. 7. Structure of fexofenadine;

FIG. 8. Reaction scheme for hydroxyl-based crosslinking with carboxylic acids;

FIG. 9. Infrared spectra of a) aspartate, b) losartan, c) polylosartan-aspartate, d) atorvastatin, e) polyatorvastatin-aspartate, f) fexofenadine, and g) polyfexofenadine-aspartate;

FIG. 10. Structure of metoprolol;

FIG. 11. Metoprolol (met)-aspartate (Asp) conjugates (left) polymerized in the presence of native (or other conjugated aspartate);

FIG. 12. Metoprolol (met)-aspartate (Asp) conjugates (left) polymerized in the absence of native (or other conjugated aspartate);

FIG. 13. Reaction scheme for hydroxyl-based crosslinking with sulfhydryls or converted free amines;

FIG. 14. Reaction scheme for hydroxyl-based crosslinking with free amines via carbonic acid, bicarbonate, diacid or multiacid;

FIG. 15. Reaction scheme for hydroxyl-based crosslinking with free hydroxyls via carbonic acid, bicarbonate, diacid or multiacid;

FIG. 16. Structure of sertraline;

FIG. 17. Reaction scheme for primary amine-based crosslinking with carboxylic acids activated by EDC;

FIG. 18. Reaction scheme for secondary amine-based crosslinking with carboxylic acids activated by EDC;

FIG. 19. Reaction scheme for primary amine-based crosslinking with carboxylic acids activated to acyl halides;

FIG. 20. Reaction scheme for secondary amine-based crosslinking with carboxylic acids activated to acyl halides;

FIG. 21. Reaction scheme for primary amine-based crosslinking with free amines via carbonic acid, bicarbonate, diacid or multiacid;

FIG. 22. Reaction scheme for secondary amine-based crosslinking with free amines via carbonic acid, bicarbonate, diacid or multiacid;

FIG. 23. Reaction scheme for primary amine-based crosslinking with free hydroxyls via carbonic acid, bicarbonate, diacid or multiacid;

FIG. 24. Reaction scheme for secondary amine-based crosslinking with free hydroxyls via carbonic acid, bicarbonate, diacid or multiacid;

FIG. 25. Structure of celecoxib (CELEBREX®);

FIG. 26. Reaction scheme for N,N unsubstituted sulfonamides based linkages to carboxylates by acylsulfonamide moieties;

FIG. 27. Structure of hydrocodone;

FIG. 28. Reaction scheme for ketone-based linkages to free amines by imine moieties;

FIG. 29. Reaction scheme for ketone-based linkages to free hydroxyl by hemi-ketal moieties;

FIG. 30. Reaction scheme for ketone-based linkages to two free hydroxyls by ketal moieties;

FIG. 31. Structure of omeprazole;

FIG. 32. Reaction scheme for activated aromatic-ring-based linkage via silicone bridges;

FIG. 33. Structure of sildenafil;

FIG. 34. Reaction scheme for reversible activation of a cyclic lactam for linkage;

FIG. 35. Structure of rofecoxib (VIOXX®);

FIG. 38. Reaction scheme for reversible activation of compounds containing a cyclic ester for linkage to an additional moiety;

FIG. 39. Reaction scheme for activation via O-acylation of pyrimidinones;

FIG. 40. Structure of clopidogrel;

FIG. 41. Structure of diltiazem;

FIG. 42. Structure of clonazepam;

FIG. 43. Structure of nifedipine;

FIG. 44. Structure of tretinoin;

FIG. 45. Structure of estradiol;

FIG. 46. Structure of levothyroxine;

FIG. 47. Structure of doxycycline hyclate;

FIG. 48. Structure of diazepam;

FIG. 49. Structure of clonidine hydrochloride;

FIG. 50. Structure of glipizide;

FIG. 51. Structure of trazodone;

FIG. 52. Structure of medroxyprogesterone acetate and progesterone;

FIG. 53. Structure of amoxicillin;

FIG. 54. Structure of methylprednisolone;

FIG. 55. Structure of allopurinol;

FIG. 56. Structure of cyclobenzaprine;

FIG. 57. Structure of albuterol sulfate;

FIG. 58. Structure of gemfibrozil;

FIG. 59. Structure of digoxin;

FIG. 60. Structure of isosorbide dinitrate.

FIG. 61; Structure of methylphenidate;

FIG. 62. Structure of octreotide acetate, (2) and the β-peptide analogue of octreotide (3);

FIG. 63. Structure of fluoxetine;

FIG. 64. Structure of lansoprazole

FIG. 65. Structure of clomiphene;

FIG. 66. Structure of amlodipine;

FIG. 67. Structure of ciprofloxacin;

FIG. 68. Structure of cefotetan;

FIG. 69. Structure of metformin;

FIG. 70. Structure of glyburide;

FIG. 71. Structure of tamoxifen;

FIG. 72. Structure of eptifibatide;

FIG. 73. Structure of pravastatin;

FIG. 74. Structure of rosuvastatin;

FIG. 75. Reaction scheme for linking to the polyaspartate backbone to codiene to form a poly-opiate;

FIG. 76. Reaction scheme for linking to the polyaspartate backbone to lorazepam to form a polyanxiolytic;

FIG. 77. Reaction scheme for linking to the polyaspartate backbone to hydrourea to form a poly-anti-cancer drug; and

FIG. 78. Reaction scheme showing the linker strategy for forming a poly-opiate.

### DETAILED DESCRIPTION OF THE PREFERRED EMBODIMENTS

Polymerized therapeutic agent compositions prepared according to the preferred embodiments of the present invention have highly desirable properties, including enhanced biological and pharmacological activities, which make them particularly well suited for use in biological and biomedical applications.

Polymerized therapeutic agent compositions can be generated in several different ways, as presented more specifically in the examples below. However, in more general terms, the polymerized compositions can generated using at least three different methods. First, the active agent can be directly linked to a polymerized backbone molecule. Second, the agent can be linked to a polymerizable backbone molecule, thereby forming a polymerizable backbone-agent conjugate. This conjugate can then be polymerized to form the complete polymerized compound. Third, the therapeutic agent can be polymerized to itself, and therefore the use of a backbone polymer or generation of a polymerizable conjugate is not required. Whatever method is used, the final polymer is made up of at least two or more active agents.

Once a polymerized therapeutic agent composition is formed, a completely unique compound with distinct physiochemical properties is obtained. For example, when compared to the original native agent, the polymerized compound will have different rates of absorption, degradation, and functionality. Likewise, by its very nature of linking together numerous actives, a polymerized compound allows for the administration of a compound with a higher per unit incorporation of a given active. This creates the added benefit of being able to focus and increase the concentration of the agent at a given target. The polymerized agent itself can be polymerized or linked to a backbone polymer wherein the linkage can be degradable or non-degradable. In its polymerized non-degradable form, the agent may be able to retain function while polymerized. In the degradable form, the active may be active in its polymerized form or inactive until processed (pro-drug). In the functional polymerized form, the active is held in a confirmation that is unique from the original non-polymerized form, thereby enhancing its activity.

The polymerized therapeutic agent compositions are preferably prepared by covalently linking subject agents to a biocompatible backbone either directly or through backbone-agent conjugates. The backbone molecule may comprise either a single molecule or a group of two or more covalently attached or otherwise associated molecules. The backbone molecule(s) should have sufficient size to carry the therapeutic agents as well as having the ability to covalently attach to other molecules. Suitable backbone polymers include poly amino acids, polyalcohols, nucleic acids, sphingosine, polysaccharides, polyacrylates, polyamines, carboxylic acids, and other homo- or copolymers with active side chains, such as carboxylates, amines, hydroxyls, amides, aromatic rings, and other hydrolyzable linkages that not only serve as binding moieties, but also can be degraded in vivo either by proteases or by non-enzymatic hydrolysis. Poly amino acids (polyaspartate and polylysine), polyalcohols (glycogen), carboxylic acids (ascorbic acid), polyacrylates, polyethlene glycol (PEG), and carbohydrates will generally be preferred as backbones for polymerization since the binding characteristics are very uniform and depend on the nature of the specific amino acid or polymer incorporated. Varying the reaction conditions can control the degree of saturation of a given agent upon a given backbone.

In additional embodiments, the functional or reactive moieties of either the backbone or agent itself can be converted using various chemical techniques to allow for different types of polymerization. Examples of such conversions or derivatives include the addition or substitution of thiol, hydroxyl, halogen, metalloids or other reactive moieties. Further, as presented below, ascorbic acid and other moieties may be bound to the therapeutic agent and remain unlinked in the final linked plurality of molecules. The unlinked ascorbic acid or other moiety will preferably retain its native activity, e.g., as an antioxidant, in the final composition.

The following examples are provided to illustrate and describe the preferred embodiments of the present invention. The scope of the invention, however, is limited only by the appended claims that follow.

Carboxylate-Based Polymerization

Examples of Polymers of Eptifibatide

For each, the monomer can include eptifibatide (refer to FIG. 72) or any analogue thereof.

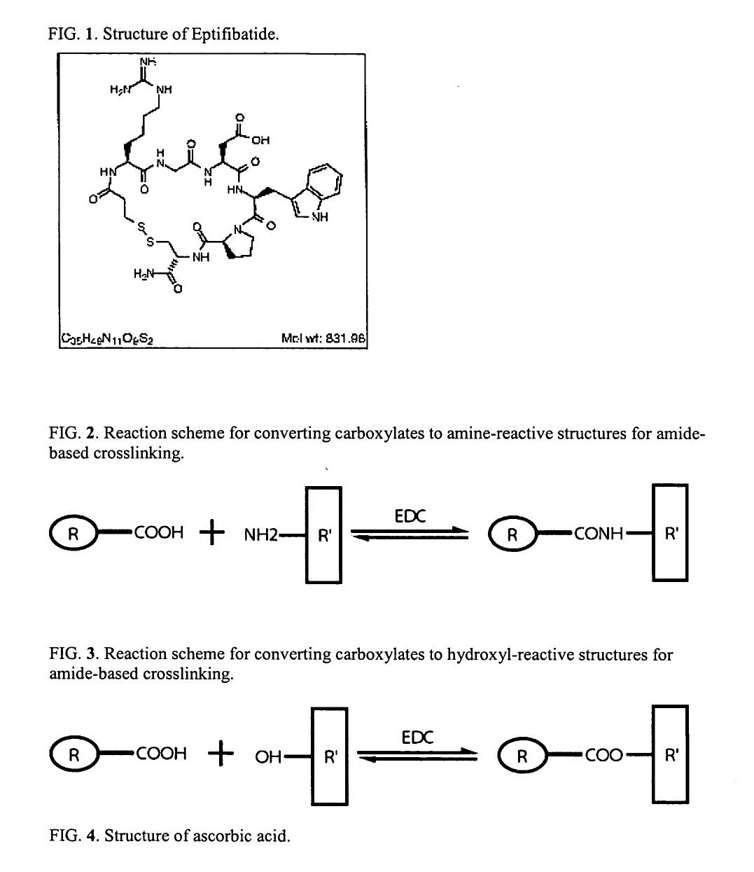

Eptifibatide (refer to FIG. 1) contains one reactive carboxylate which can be used to form polymers as described in examples below.

Polylysine with Eptifibatide Side Chains.

Activated carboxylates can be made amine-reactive as outlined in FIG. 2. Polylysine (p-1399, Sigma Chemical Company, St. Louis, Mo.) has free primary amines as termini on each side chain. Using an activating agent such as EDC (Pierce Endogen, Rockford, Ill.), the carboxylate of eptifibatide can be made amine reactive and coupled with the free side chain amine of lysine (then polymerized as before) or of polylysine. The former requires standard protection of the N terminus. The resulting product would have degradable peptide linkages and degradable side chain amides. Alternately, eptifibatide can be reacted with lysine directly, and the resulting compound can be polymerized via amide linkages as will be readily apparent to one skilled in the art.

Eptifibatide Bound to a Polyol Backbone.

Activated carboxylates can be made hydroxyl-reactive as outlined in FIG. 3. The carboxylate of eptifibatide can be activated under acidic conditions or via chemical activating agent to form esters via free hydroxyls on glycogen or other polyol. The polyol can be linear or branched as desired. The resulting ester linkages are degradable in aqueous environments under physiologic conditions.

Polymerized Eptifibatide Ascorbic Acid Conjugates.

Eptifibatide is reacted with ascorbic acid (structure in FIG. 4) to produce an ester linkage according to the method presented above or other well-known techniques; see U.S. Patent Publication Nos. U.S. 2002/0031557 A1; US 2002/0037314 A1; and U.S. 2001/0041193 A1; and Maugard, T., et al. (2000). Studies of vitamin ester synthesis by lipase-catalyzed transesterification in organic media. Biotechnol. Prog. 16(3):358-362. The eptifibatide-ascorbic acid conjugates are then polymerized via free hydroxyls on the ascorbic acid or anchored to a polymerizable backbone using the techniques described above (under “Eptifibatide bound to a polyol backbone”). Ascorbic acid, also known as vitamin C, is an anti-oxidant which may provide benefits when compositions according to the present invention are used for hyperplasia inhibition or other purposes. Remaining free hydroxyls can be derivatized or reacted to add polymerizable groups. Free acid groups can react with hydroxyls from adjacent hybrids to cross-link directly or can be reacted with a separate backbone.

Eptifibatide with Other Materials.

Eptifibatide may be derivatized with other materials, which are useful for polymerization and which also provide other functionalities in the polymerized molecules For example, eptifibatide may be derivatized with vitamin E, various nitric oxide donors, anti-angiogenic agents, such as angiostatin, HMG-CoA reductase inhibitors, and the like. The resulting heterobifunctional (or heteromultifunctional) eptifibatide monomers may then be polymerized to produce compositions according to the present invention using known techniques.

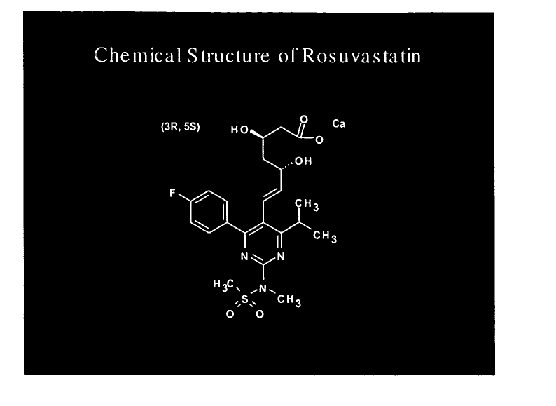

Other agents suitable for carboxylate-based polymerization include but are not limited to: fexofenadine, infliximab, atorvastatin, trastuzumab, cefotetan, gadopentate, LU135252 (a selective antAGO-nist of the ETA receptor), omapatrilat, neotrophin, c-peptide, cerebrolysin, pentfuside, PRO542 (a recombinant heterotetrameric fusion protein), VEGF121 (Vascular Endothelial Cell Growth Factor FORM 121), C₁₋₁₀₂₃ (VEGF adenovirus), FGF2 (Fibroblast growth factor 2), neutralase, rNAPc2 (recombinant Nematode Anticoagulant Protein c2), natrecor, bivalarudin, TP-10 Immunotherapeutics, entanercept, teneceplase, recombinant ApoA-1 Milano protein (apo a-1-Milano), AGO-1067 (an atherosclerosis drug), heparin, rosuvastatin (refer to FIG. 74), NK-104 (superstatin pitavastatin), liprostin, argatroban, abciximab, ibuprofen, naproxen, RSR13 (efaproxiral, a synthetic allosteric modifier of hemoglobin), atacand candesartan, valsartan, YM872 (a water-soluble á-amino-3-hydroxy-5-methylisoxazole-4-propionic), lisinopril, furosemid, amoxicillin, captopril, and doxazosin.

Hydroxyl-Based Polymerization

Examples of Polymers of Losartan, Pravastatin, Atorvastatin, and Fexofenadine

Drug Stock Solutions:

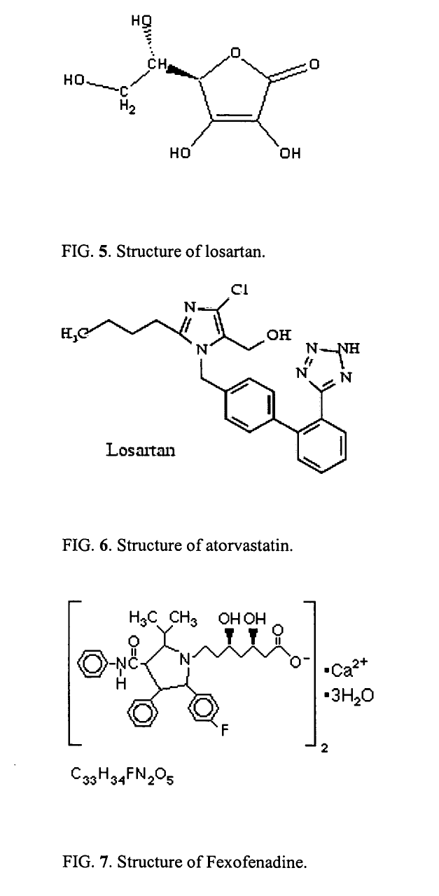

Stock solutions of losartan (refer to FIG. 5), atorvastatin (refer to FIG. 6), and fexofenadine (refer to FIG. 7) were prepared by soaking pills in water to remove gelatin coat, swirling, decanting, and patting dry with a paper towel. This was done twice with care taken to avoid too much dissolution/suspension of the pill substance. The approximate weight of the uncoated pills was noted, and then the pills were crushed to dust in a mortar and pestle. The dust was weighed, and divided by the weight of a single (coated) pill. This figure was then multiplied by the amount of drug per pill to yield the approximate weight of drug. The pill dusts were added to N,N dimethyl formamide (DMF): water (1:1, by volume) and stirred for at least 2 hours. After this, each solution was centrifuged first at 1250 time gravity (x g) for 10 min with decantation of liquid phase, which underwent additional centrifugation at 10000 rpm in the Eppendorf centrifuge for 5 minutes; stock solution is the liquid phase. The stock solution of atorvastatin in the polyaspartate experiments was prepared as above except the coating was not dissolved off of the pills. (ref. CBII p. 4)

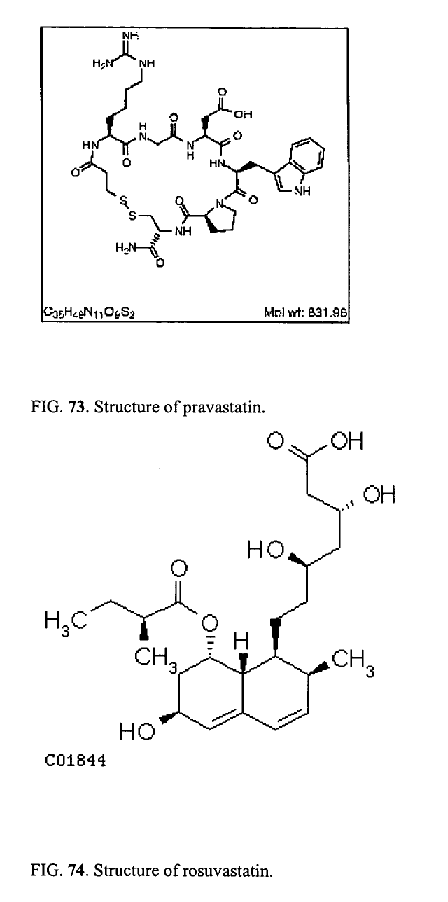

Polyaspartate with Pravastatin (Refer to FIG. 73), Atorvastatin, or Fexofenadine Side Chains.

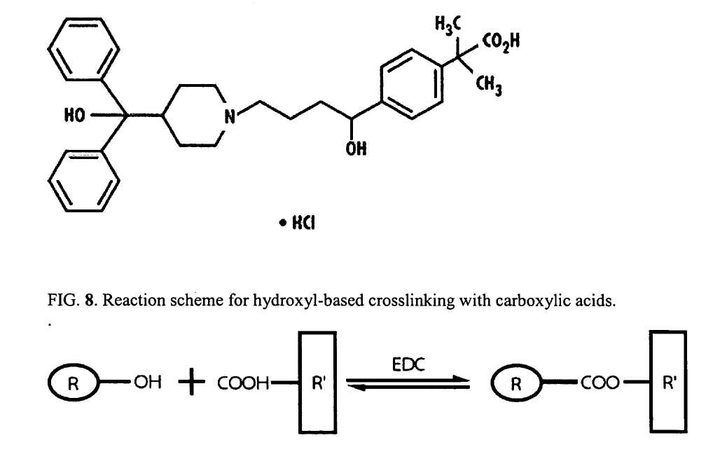

Hydroxyls can be reacted with activated carboxylic acids to form ester linkages as summarized in FIG. 8.

General Procedure: To 1.0 ml of 0.1 M MES buffer, pH 5.0, was added sodium polyaspartate, FW ˜30000 amu (0.05 or 0.2 ml, depending on the sample) as a 50 mg/ml solution in water. A portion of the buffered polyaspartate solution was then used to wash dry EDC (16.6 mg, 0.087 mmol, 260 eq) out of a pre-weighed container, and then placed back into the original incubation mix with stirring. Drug solutions, (see above), approx 3.4 ml each, (300 eq.), as well as a control consisting of 1:1 DMF: water (3.4 ml) were then added to the individual activated polymer solutions. Finally, DMAP (0.01 ml, cat) was added as a 50 mg/ml solution in DMF. Reaction pH was found to be ˜7 at start. The reactions were allowed to stir overnight at room temperature. Some of the reactions (Fexofenadine and Atorvastatin) turned cloudy to slightly cloudy almost immediately.

Workup: Separation of the putative polyaspartate-drug adducts was accomplished using a Microsep 10K Omega membrane filter (Pall Corp, Ann Arbor, Mich.). According to manufacturer's verbally conveyed information, such filters are able to operate as advertised in a solution of 40% DMF in water. To be safe, our reactions were diluted to 28% by addition of water as follows.

From each reaction (vol ˜4.4 ml) was removed 3 ml and this was added to 3.5 ml water. This 6.5 ml of diluted reaction mixture was placed atop the centrifugation membrane then centrifuged at 4° C. for 0.5 hrs and 1250×g. The samples were then centrifuged at 3200×g until volume remaining above the membrane was less than 1.5 ml. The samples were than washed with 3 ml water, centrifugation repeated. Two of the samples (atorvastatin and fexofenadine) required several rounds of stirring and centrifugation to accomplish separation and washing. The atorvastatin sample was centrifuged to a final volume (after washing) of ˜2 ml The fexofenadine sample was placed in a new concentrator and centrifuged until less than 1.5 ml, washed with 3 ml water, and centrifuged down to around 3 ml. (ref: CBII, p. 8-9).

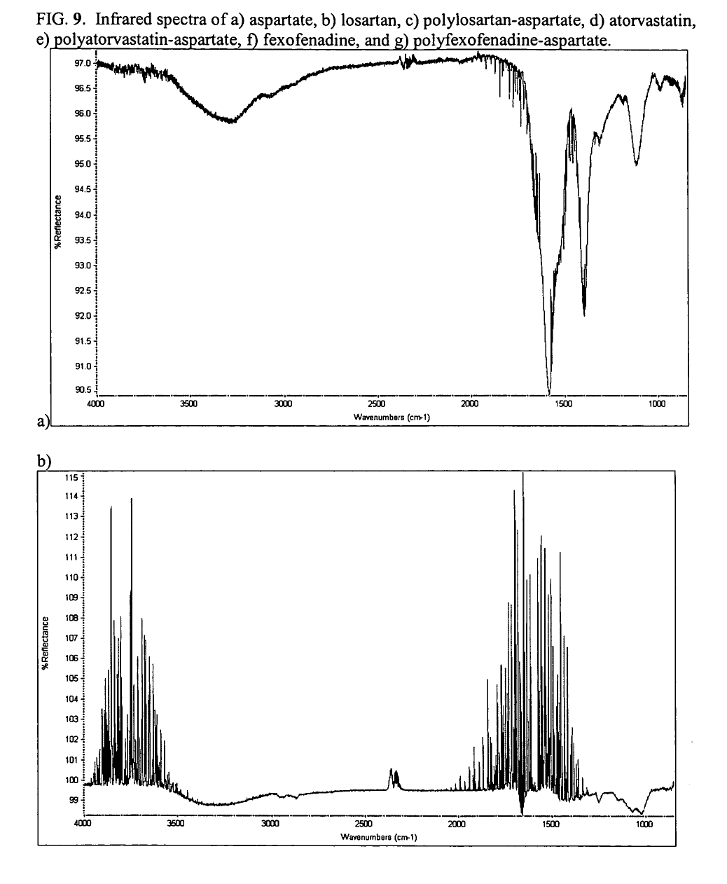

Data Summary: Please refer to FIG. 9. IR of polyaspartate alone exhibits free acid peaks (from side chains) and amide peaks (from backbone). After incubation with pravastatin, atorvastatin, or fexofenadine, the IR absorbance of the free acid declines progressively and a new ester peak begins to appear and increase in signal. As degree of saturation increases, the peaks become comparable in intensity (as assessed by peak integration). Number of polymerized moieties can thus be controlled and monitored by sequential activation. Alternately, the monomer of interest (pravastatin, atorvastatin, and fexofenadine) could have been anchored to aspartic acid and the resulting product cross-linked directly, as will be readily apparent to one skilled in the art.

To demonstrate that the linkage was not peptide dependant, comparable linkage to acrylates was undertaken as follows:

Polyacrylate with Pravastatin, Atorvastatin, or Fexofenadine Side Chains.

General Procedure: To 1.0 ml; of 0.1 MES buffer, pH 5.0 was added Polyacrylate (Luvigel, BASF corp., 25% emulsion in water) 0.01 ml. This mixture was then used to wash dry EDC (16.6 mg, 0.087 mmol 260 eq) out of a pre-weighed container, and then placed back into the original incubation mix with stirring. Added drug stock solution (300 eq, or control, all ˜3.4 ml in 1:1 DMF: water). Stir, then add DMAP (0.5 mg, cat) as a 50 mg mg/ml solution in DMF. Reaction pH was taken at 45 min and found to be ˜5. All of the reactions appeared to produce solid residue. They were stirred for 18 hours at room temperature. (ref: CBII, p. 13, 14).

Workup: The reactions were stopped from stirring. The presence of solid material was noted. A 1 ml aliquot (including solid) was removed from each, and centrifuged at 10,000 rpm for 10 min in the Eppendorf benchtop device. The supernatant was decanted and saved, and DMF (0.5 ml) then water (0.5 ml) was added to each tube, vortexing for 30 sec. Centrifuged at 14000 rpm for 15 min, collected the precipitate, and sent it for IR analysis. (ref: CBII, p. 15).

Data Summary: IR data was taken for samples CBII-15. Fexofenadine showed flattening of the acid peaks (2) along with the emergence of 2 new peaks in the ester region. Atorvastatin showed some ester peak, but intermediate intensity. Cozaar was most resistant to coupling (ester).

Examples of Polymers of Metoprolol

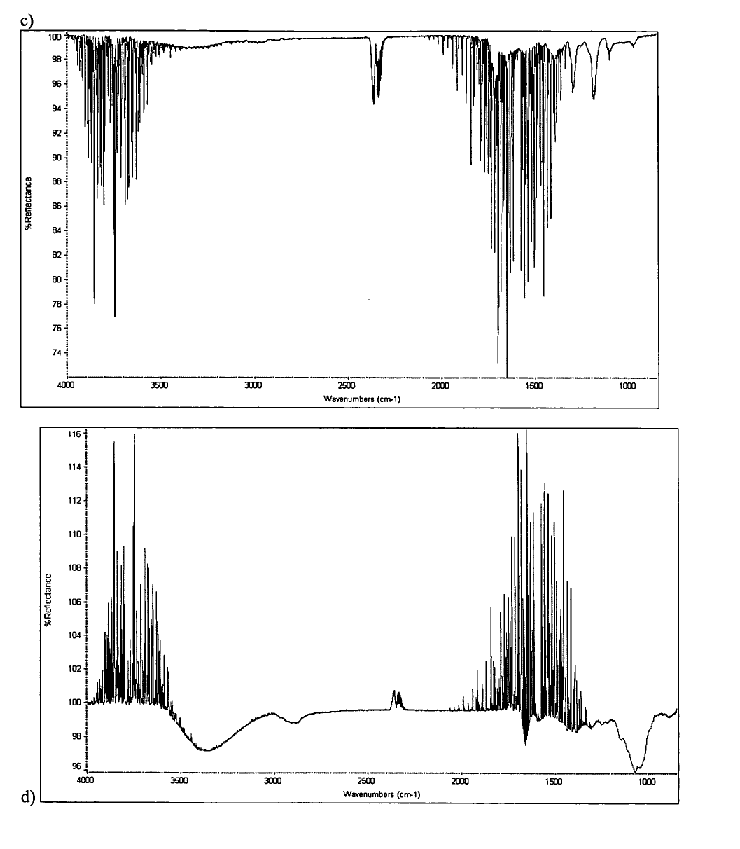

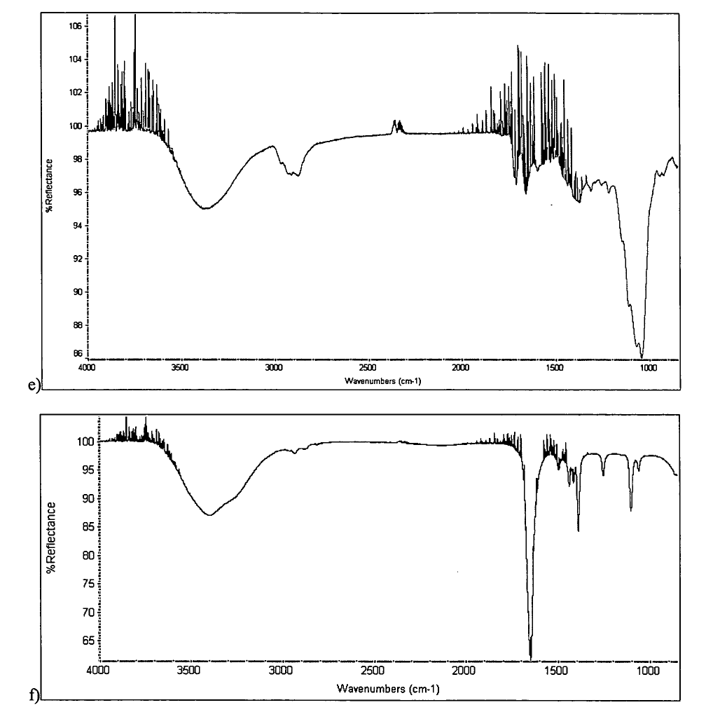

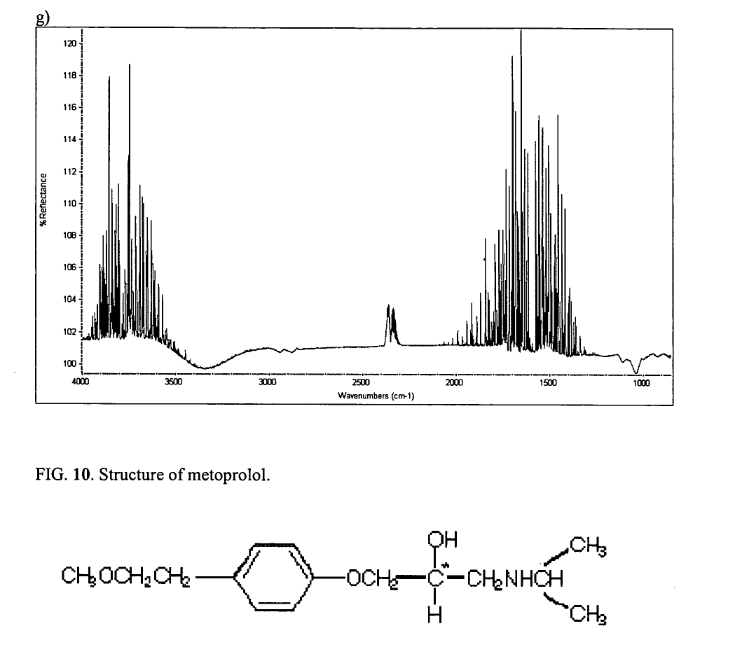

For each, the monomer can include metoprolol, or any analogue thereof. Metoprolol (refer to FIG. 10) contains a reactive hydroxyl which can be used to form polymers as described in examples below.

1. Polyaspartate with Metoprolol Ester Side Chains.

Please refer to FIG. 8 for general scheme of reacting hydroxyls with activated carboxylic acids. Sodium polyaspartate (Aquadew SPA-30, Ajinomoto, Tokyo, Japan) is reacted with metoprolol in the presence of a sulfuric acid catalyst using standard methods; see De Carvalho, M. G. S. et al., Identification of phosphorylation sites of human 85-kda cytosolic phospholipase A2 expressed in insect cells and present in human monocytes, 1996. J. Biol. Chem 271(12):6987-97. Free carboxylic acid termini on the sodium polyaspartate react with the free hydroxyl of metoprolol to form an ester linkage which is degradable in aqueous environments under physiologic conditions. Additionally, the amide linkages of the polyaspartate backbone can be degraded in vivo either by proteases or by non-enzymatic hydrolysis. In this way, multiple metoprolols are added to a single backbone of the polyaspartate. The degree of saturation of metoprolol on the polyaspartate can be controlled by varying the reaction conditions, such as the concentration of metoprolol, the concentration of sodium polyaspartate, the concentration of the sulfuric acid catalyst, the duration of the reaction, the temperature of the reaction, and the like, as is well-known to one skilled in the art.

2. Polyaspartate Having Metoprolol Ester Side Chains.

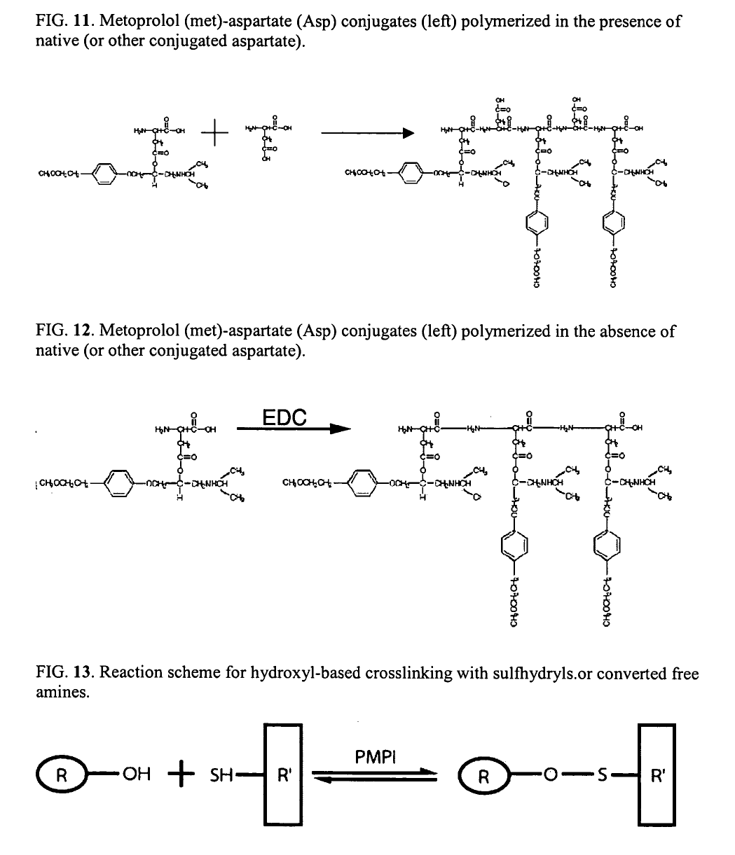

Polyaspartate having metoprolol ester side chains could also be formed by first forming metoprolol esters with aspartate monomers. The metoprolol ester aspartate monomers could then be polymerized by forming amide linkages between the aspartates. The number of metoprolols incorporated in each polyaspartate form can be controlled by reacting the metoprolol derivatized aspartates with native or otherwise derivatized aspartates. Metoprolol (met)-aspartate (Asp) conjugates may be polymerized in the presence of (refer to FIG. 11) or absence (refer to FIG. 12) of native (or other conjugated aspartate). The ratio of native to conjugated aspartate in the polymer will be the same as that in the reaction volume, so the degree of metoprolol saturation in the resulting polymer can be determined in the protocol of FIG. 11.

3. Polylysine with Metoprolol Side Chains.

Hyrdoxyls can be reacted with sulfhydryl groups using standard linkers as described below. Since primary amines can be converted to sulfhydryls, these methods provide a scheme for linking hydroxyl-containing compounds to linkable moieties with either free amines or sulfhydrils as summarized in FIG. 13. Polylysine (p-1399, Sigma Chemical Company, St. Louis, Mo.) has free primary amines as termini on each side chain. The free primary amines are converted to free thiols using Traut's reagent (Pierce Endogen, Rockford, Ill.) under standard conditions. The reaction can be controlled to convert any number of the side chain amines from a minimum of three to all. The thiol side chains are then covalently bound to the free hydroxyl of metoprolol using PMPI (Pierce Endogen), according to the manufacturer's recommendations. PMPI is a heterobifunctional linker which joins free hydroxyls and free thiols. The PMPI linker could be used with other poly (amino acids) or polypeptides which have free thiols in their side chains.

4. Polylysine with Amide-Ester Link Metoprolol.

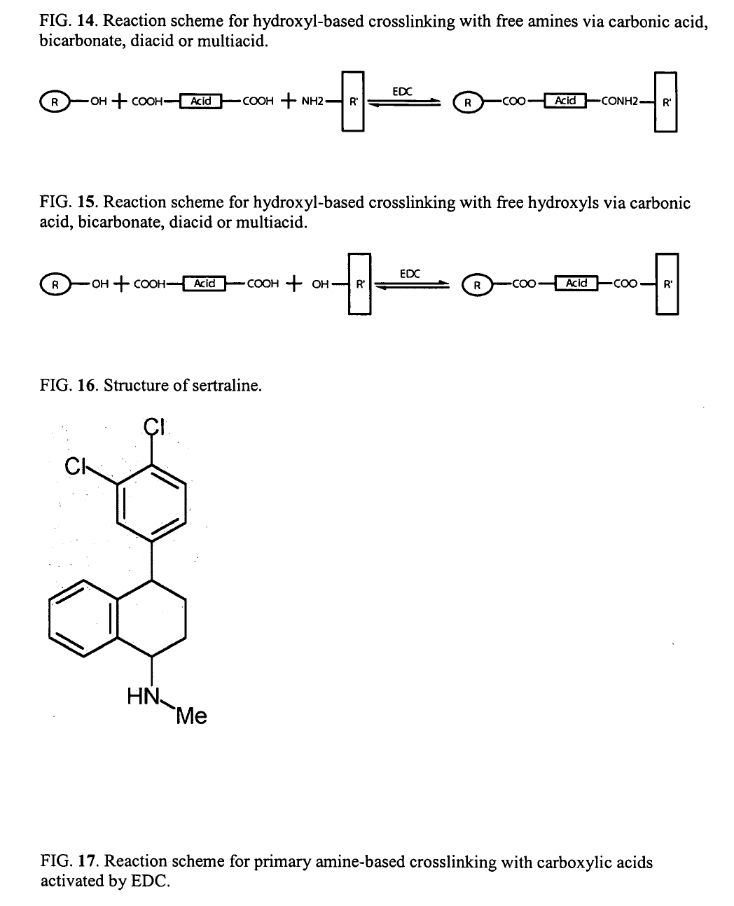

The free amines of polylysine are reacted with the free hydroxyl of metoprolol using carbonic acid, bicarbonate, diacid or multiacid. This reaction is described in U.S. Pat. No. 6,371,975 and here generates a mixed polymer of metoprolol and a free amine-rich peptide with mixed ester-amide linkages (refer to FIG. 14). The ester-amide linkages are degradable.

5. Metoprolol Bound to a Polyol.

As described in U.S. Patent Publication No. US 2002/0055518A1, free thiols can be generated on metoprolol. The free thiols on the metoprolol may then be reacted with a linear or branched polyol such as glycogen to produce a composition according to the present invention using a linker such as PMPI which joins free hydroxyls and sulfhydryls (following FIG. 13). Alternately, carbonic acid, bicarbonate, diacid (or multi-acid) can be used to form a mixed ester between the hydroxyls of metoprolol and the hydroxyls of a polyol (refer to FIG. 15) using methods described in U.S. Pat. No. 6,371,975. The resulting mixed diester linkages are degradable in aqueous environments under physiologic conditions.

6. Polymerized Metoprolol Ascorbic Acid Conjugates.

Metoprolol is reacted with ascorbic acid to produce an ester linkage according to well-known techniques; see U.S. Patent Publication Nos. U.S. 2002/0031557 A1; U.S. 2002/0037314 A1; and U.S. 2001/0041193 A1; and Maugard, T., et al. (2000). Studies of vitamin ester synthesis by lipase-catalyzed transesterification in organic media. Biotechnol. Prog. 16(3):358-362. The metoprolol ascorbic acid conjugates are then polymerized via free hydroxyls on the ascorbic acid and/or metoprolol or anchored to a polymerizable backbone using the techniques described above. Ascorbic acid, also known as vitamin C, is an anti-oxidant which may provide benefits when compositions according to the present invention are used for hyperplasia inhibition or other purposes. Remaining free acid groups can react with hydroxyls from adjacent hybrids to cross-link directly or can be reacted with a separate backbone.

Metoprolol may be derivatized with other materials which are useful for polymerization and which also provide other functionalities in the polymerized molecules. For example, metoprolol may be derivatized with vitamin E, various nitric oxide donors, anti-angiogenic agents, such as angiostatin, HMG-CoA reductase inhibitors, and the like. The resulting heterobifunctional metoprolol monomers may then be polymerized to produce compositions according to the present invention using known techniques.

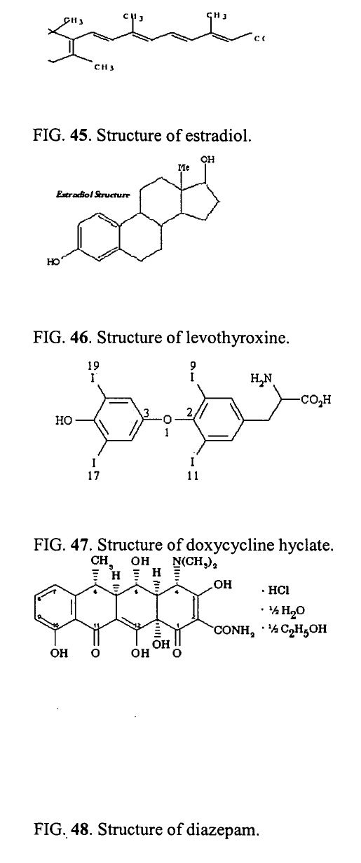

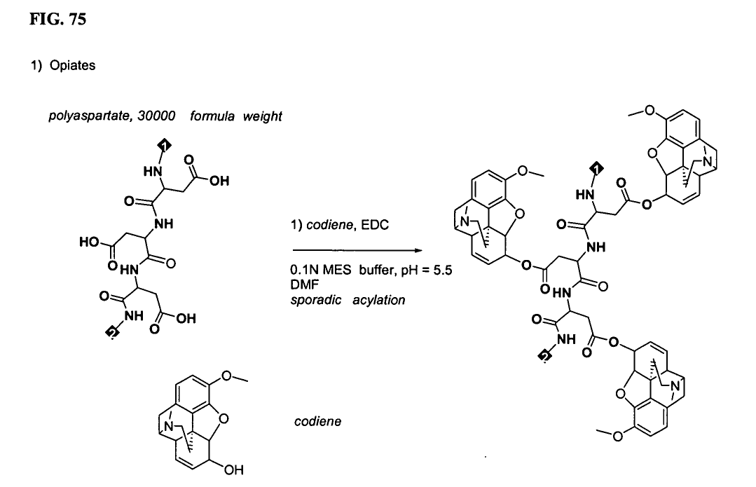

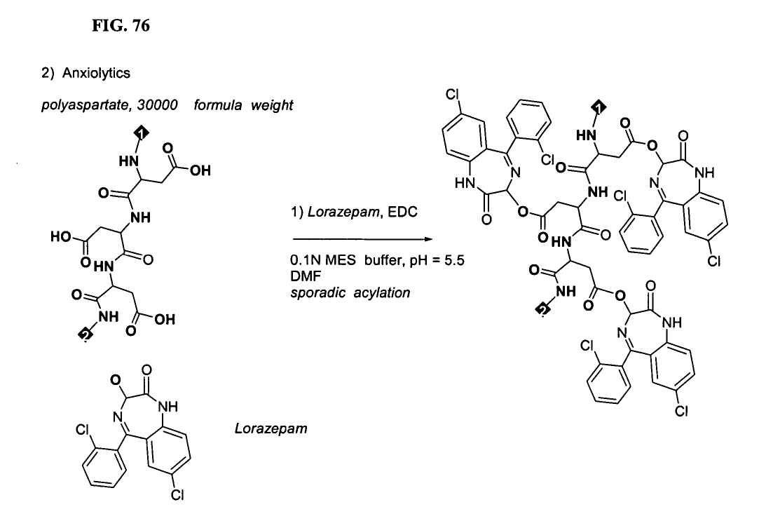

Other agents suitable for hydroxyl-based polymerization include but are not limited to: octreotide (refer to FIG. 62), infliximab, trastuzumab, LU135252, BMS-232623, tecadenoson, c-peptide, cerebrolysin, pentfuside, PRO542, VEGF121, CI-1023, FGF2, neutralase, rNAPc2, natrecor, bivalarudin, tp10, entanercept, teneceplase, apo a-1-Milano, AGO-1067, heparin, rosuvastatin, NK-104, liprostin, TBC3711 (a therapeutic agent developed by ICOS Corporation), hydroxyurea, emtricitabine, citicoline, DAPD (2,6-diaminopurine, a dioxolanyl nucleoside analogue), carvedilol, osycodone, hydromorphone, calanolide a, mycophenylate, tipranavir, ranolazine, tracleer bosentan actelion, tezosentan, santidar fondiparinux, pkc (protein kinase c) inhibitor, angiogenix, motefaxin lutetium, azithromycin, atenolol, albuterol, propoxyphene, prednisone, lorazepam, temaxepam, warfarin, estradiol (refer to FIG. 45), doxycycline, codiene (refer to FIG. 75), morphine, oxymorphone, and endomorphone.

Amine-Based Polymerization

Examples of Polymers of Sertraline

For each, the monomer can include sertraline (refer to FIG. 16), or any analogue thereof. Sertraline contains a reactive nitrogen which can be used to form polymers as described in examples below.

Polyaspartate with Sertraline Amide Side Chains.

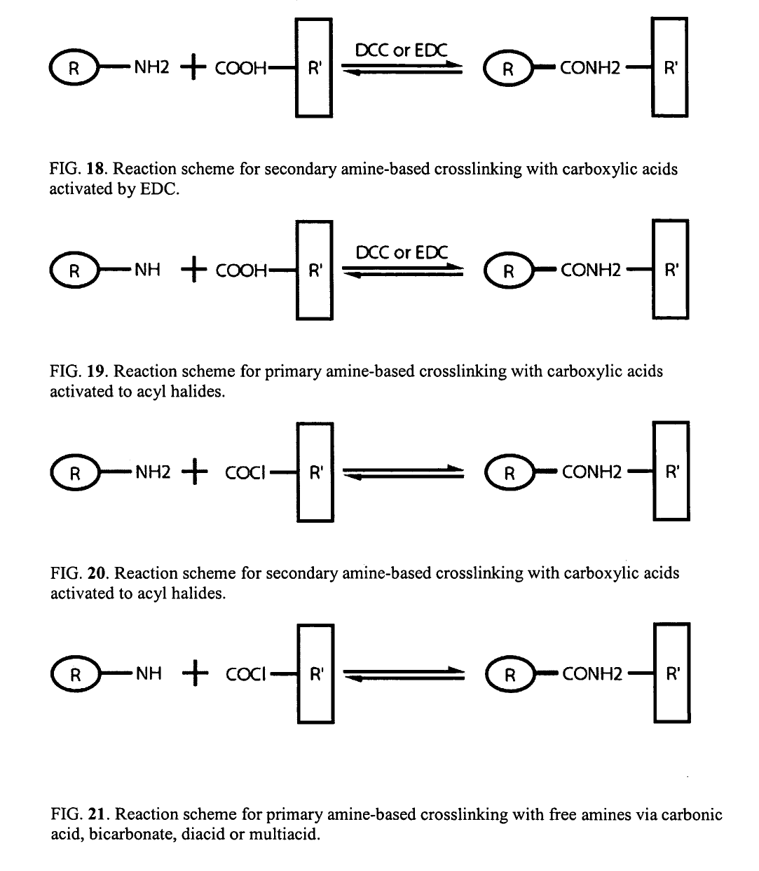

Free amines can be reacted with activated carboxylic acids to form amide linkages with linkable side moieties as depicted in FIG. 17 for primary amines and FIG. 18 for secondary amines. Sodium polyaspartate (Aquadew SPA-30, Ajinomoto, Tokyo, Japan) is reacted with sertraline. Free carboxylic acid termini on the sodium polyaspartate react the secondary amine of sertraline to form a carboxylate-ammonium salt that can be pyrolyzed to give an amide linkage which is degradable in aqueous environments under physiologic conditions; see Mitchell, J. A.; Reid, E. E. J. Am. Chem. Soc., 1931, 53, 1879; Jursic, B. S.; Zdravkovski, Z. Synth. Commun., 1993, 23, 2761. The reaction between the carboxylic acid termini on the sodium polyaspartate and the secondary amine on sertraline can also be made to proceed by the use of coupling agents, such as DCC, (Klausner, Y. S.; Bodansky, M. Synthesis, 1972, 453.) or an activating agent such as EDC (Pierce Endogen, Rockford, Ill.) that makes the caboxylates polyaspartate reactive toward the secondary amine of sertratline. Additionally, the amide linkages of the polyaspartate backbone can be degraded in vivo either by proteases or by non-enzymatic hydrolysis. In this way, multiple sertralines are added to a single backbone of the polyaspartate. The degree of saturation of sertraline on the polyaspartate can by controlled by varying the reaction conditions, such as the concentration of sertraline, the concentration of sodium polyaspartate, the duration of the reaction, the temperature of the reaction, and the like, as is well-known to one skilled in the art. Polyaspartate having sertraline amide side chains could also be formed by first forming sertraline amides with aspartate monomers. The sertraline amide aspartate monomers could then be polymerized by forming amide linkages between the aspartates. The number of sertralines incorporated in each polyaspartate form can be controlled by reaction the sertraline derivatized aspartates with native or otherwise derivatized aspartates. The ratio of native to conjugated aspartate in the polymer will approximate that in the reaction volume, so the degree of sertraline saturation in the resulting polymer can be determined in the protocol.

Alternately, the carboxylic acids of polyaspartate are converted to amine-reactive acyl halides using phosphorus tribromide or SOCl₂ under standard conditions as depicted in FIG. 19 for primary amines and FIG. 20 for secondary amines. The reaction can be controlled to convert any number of the side chain carboxylates from a minimum of three to all. The acyl halide side chain then reacts with the secondary amine of sertraline to form an amide linkage which is degradable in aqueous environments under physiologic conditions; see Jedrzejczak, M.; Motie, R. E.; Satchell, D. P. N. J. Chem. Soc., Perkin Trans. 2, 1993, 599. The resulting product would also have degradable peptide linkages.

Polylysine with Amide Linked Sertraline.

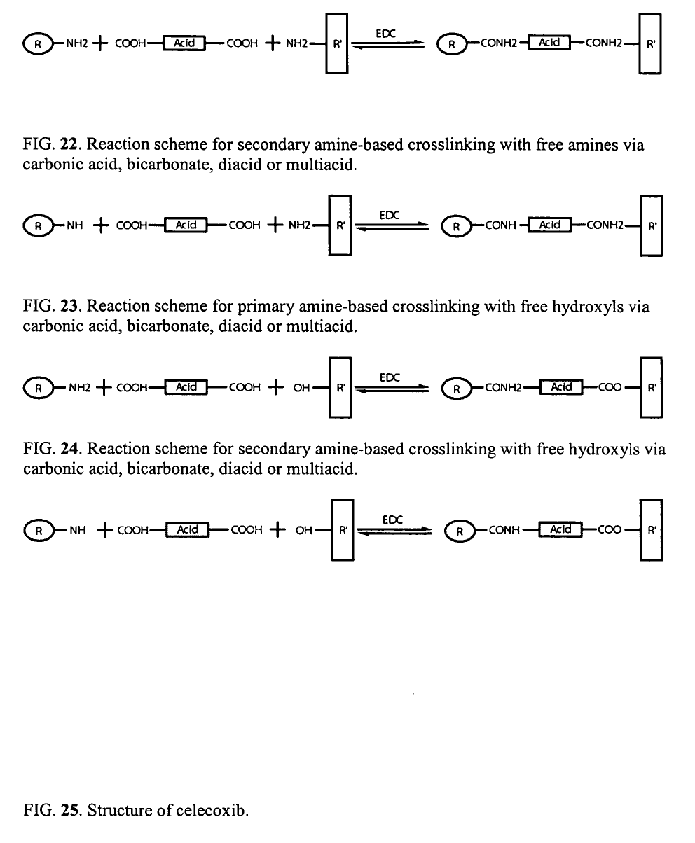

The free amines of polylysine are reacted with a primary (refer to FIG. 21) or secondary (refer to FIG. 22) amine such as sertraline using carbonic acid, bicarbonate, diacids, or multiacids. This reaction is described in U.S. Pat. No. 6,371,975 and generates a mixed polymer of sertraline and a free amine-rich peptide with amide linkages. The amide linkages are degradable.

Sertraline Bound to a Polyol Backbone.

Carbonic acid, bicarbonate, diacids, or multiacids can be used to form mixed ester-amide linkages between the hydroxyls of a linear or branched polyol such as glycogen and a primary (refer to FIG. 23) or secondary (refer to FIG. 24) amine such as sertraline using methods described in U.S. Pat. No. 6,371,975.

Polymerized Sertraline Ascorbic Acid Conjugates.

Sertraline is reacted with ascorbic acid to produce an amide linkage according to well-known techniques; see U.S. Patent Publication Nos. U.S. 2002/0031557 A1; U.S. 2002/0037314 A1; and U.S. 2001/0041193 A1; and Maugard, T., et al. (2000). Studies of vitamin ester synthesis by lipase-catalyzed transesterification in organic media. Biotechnol. Prog. 16(3):358-362. The sertraline-ascorbic acid conjugates are then polymerized via free hydroxyls on the ascorbic acid or anchored to a polymerizable backbone using the techniques described above. Ascorbic acid, also known as vitamin C, is an anti-oxidant which may provide benefits when compositions according to the present invention are used for hyperplasia inhibition or other purposes. Remaining free hydroxyls can be derivatized or reacted to add polymerizable groups and free acid groups can react with hydroxyls from adjacent hybrids to cross-link directly or can be reacted with a separate backbone.

Sertraline may be Derivatized with Other Materials

Sertraline may be derivatized with other materials which are useful for polymerization and which also provide other functionalities in the polymerized molecules. For example, sertraline may be derivatized with vitamin E, various nitric oxide donors, anti-angiogenic agents, such as angiostatin, HMG-CoA reductase inhibitors, and the like. The resulting heterobifunctional (or heteromultifunctional) sertraline monomores may then be polymerized to produce compositions according to the present invention using known techniques.

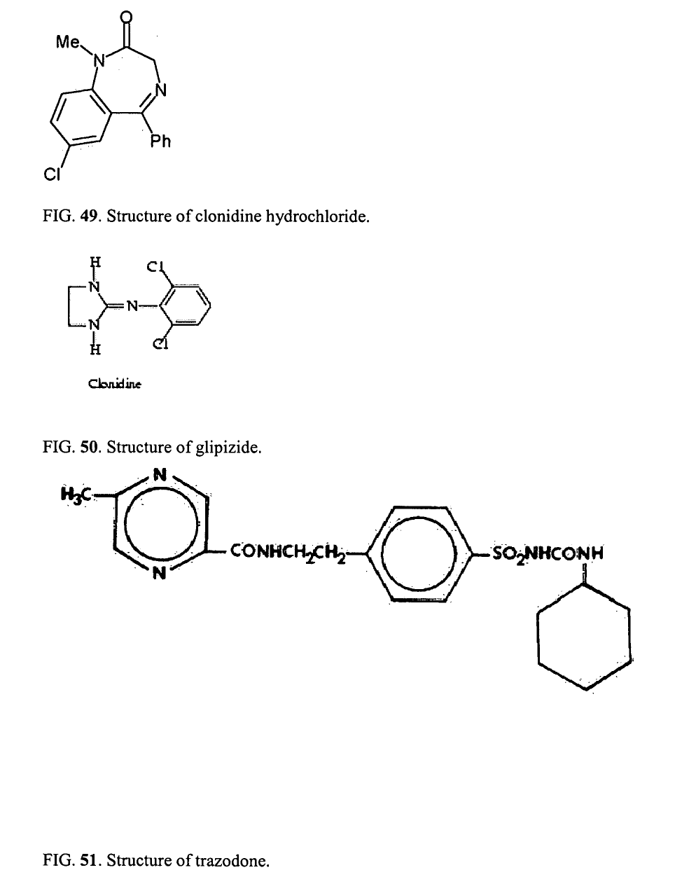

Other agents suitable for amine-based polymerization include but are not limited to: methylphenidate, metroprolol, octreotide, fluoxetine, infliximab, atorvastatin, amlodipine, ciprofloxacin, trastuxumab, esomeprazole, omeprazole, metformin, eptifibatide, gadopentate, neotrophin, c-peptide, cerebrolysin, pentfuside, PRO542, VEGF121, C₁₋₁₀₂₃, FGF2, neutralase, rNAPc2, natrecor, bivalarudin, TP-10, entanercept, teneceplase, apo a-1-Milano, argatroban, abciximab, lisinopril, hydroxyurea, emtricitabine, citicoline, DAPD, carvedilol, capraverine, cariporide, niaspan, ADA, tmcl25, huperzine q, panzem tmcl20, atenolol, furosemide, triamterene, ranitidine, albuterol, amoxicillin, propoxyphene, fluoxetine, doxazosin, sulfamethoxazole, trimetrhoprim, nifedipine (refer to FIG. 43), and clonidine (refer to FIG. 49).

Sulfonamide-Based Polymerization

Examples of Polymers of N,N Unsubstituted Sulfonamides

Drugs containing N,N unsubstituted sulfonamides are linked to polymer by acylsulfonamide linkages as follows:

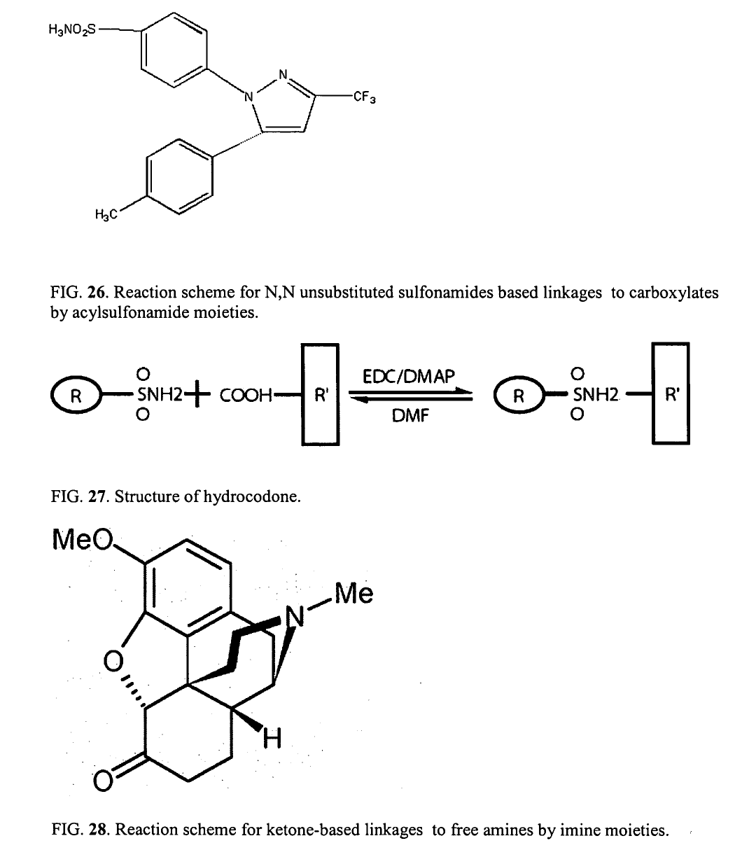

Using celecoxib as an example (structure, FIG. 25; reaction scheme FIG. 26): To DMF is added Sodium Polyaspartate, FW 30000 amu. (10 mg, as a solid). A DMF solution of EDC (16.6 mg, 0.087 mmol, 260 eq) is added to the suspended solid with stirring. Celecoxib, approximately 300 eq., is added. Finally, DMAP (0.01 ml, cat) was added as a 50 mg/ml solution in DMF. The reactions are allowed to stir overnight at room temperature. The samples are diluted with water (to 25% DMF by volume) and worked up as before. Ref: Biorg. Med. Chem. Lett.: 2001:2355.

Other agents suitable for sulfonamide-based polymerization include but are not limited to: sertraline, metformin, rosuvastatin, argatroban, TBC 1711, tipranavir, tracleer bosentan actelion, tezosentan, xantidar fondiparinux, cariporide, VX-175 (an HIV protease inhibitor), BMS-207940 (a biphenylsulfonamide endothelin A receptor-selective antagonist), rofecoxib, furosemide, glyburide, and sulfamethoxazole.

Ketone-Based Polymerization

Examples of Polymers of Hydrocodone

For each, the monomer can include hydrocodone (refer to FIG. 27), or any analogue thereof. Hydrocodone contains a reactive ketone carbonyl which can be used to form polymers as described in examples below.

Polylysine with Hydrocodone Side Chains.

Polylysine (p-1399, Sigma Chemical company, St. Louis, Mo.) has primary amines as termini on each side chain. The free amines on polylysine react with the ketone carbonyl on hydrocodone to form stable imine linkages that are degradable in aqueous environments under physiologic conditions as depicted in FIG. 28. Dayagi, S.; Degani, Y. The Chemistry of the Carbon-Nitrogen Double Bond. Additionally, the amide linkages of the polylysine backbone can be degraded in vivo either by proteases or by non-enzymatic hydrolysis. In this way, multiple hydrocodones are added to a single backbone of the polylysine. The degree of saturation of hydrocodone on the polylysine can be controlled by varying the reaction conditions, such as the concentration of hydrocodone, the concentration of polylysine, the concentration of the catalyst, the duration of the reaction, the temperature of the reaction, and the like, as is well-known to one skilled in the art.

Polylysine having hydrocodone imine side chains could also be formed by first forming hydrocodone imines with lysine monomers. The hydrocodone imine lysine monomers could then be polymerized by forming amide linkages between the lysines. The number of hydrocodones incorporated in each polylysine form can be controlled by reacting the hydrocodone derivatized lysines with native or otherwise derivatized lysines. The ratio of native to conjugated lysine in the polymer will approximate that in the reaction volume, so the degree of hydrocodone saturation in the resulting polymer can be determined in the protocol.

Hydrocodone Bound to a Carbohydrate Backbone.

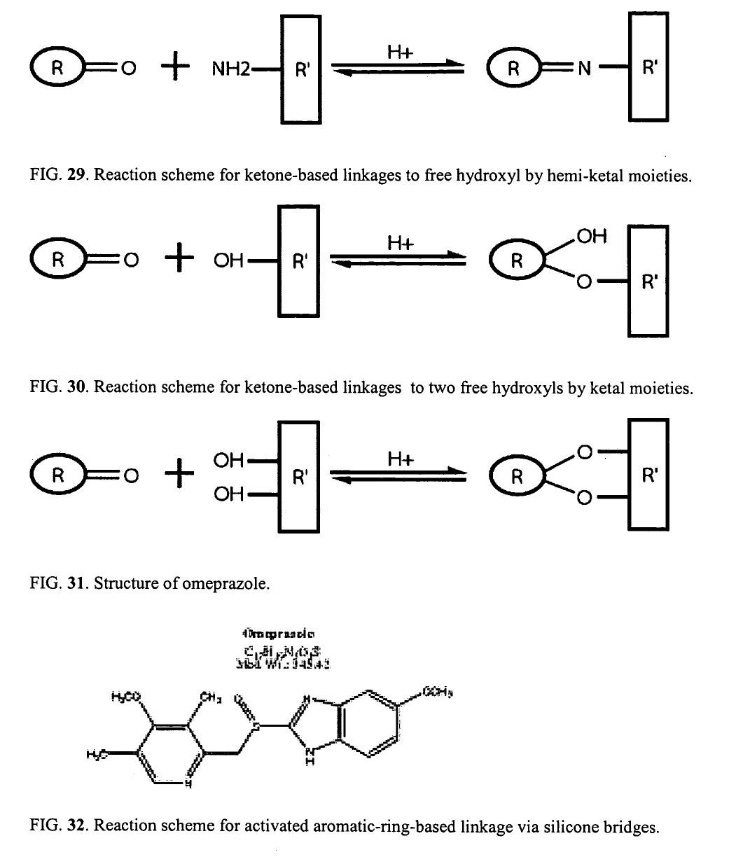

One free hydroxyl on a carbohydrate will react with the ketone carbonyl of hydrocodone to produce a hemiketal (refer to FIG. 29) link under acidic conditions. Two proximally close hydroxyls on the carbohydrate will react with the single ketone carbonyl of hydrocodone to produce a ketal (refer to FIG. 30) linkage under acidic conditions; see Meskens, F. A. J. Synthesis, 1981, 501; and Schmitz, E.; Eichhom, I. in Patai, The Chemistry of the Ether Lingkage [sic]; Wiley: NY, 1967, p. 309. Both the hemiketal and ketal links will readily hydrolyze in an aqueous environment under physiologic conditions to regenerate the carbohydrate and the hydrocodone. Suitable carbohydrates will have no less than six hydroxyl groups each. The resulting n-hydro-codone carbohydrates can also polymerize under the conditions described above. The ketone or aldehyde carbonyl of the carbohydrate reacts with one or two free hydroxyls of another n-hydrocodone carbohydrate to form a hemiketal or ketal link, respectively, forming a multi-hydrocodone multi-carbohydrate polymer that is degradable in aqueous environments under physiologic conditions.

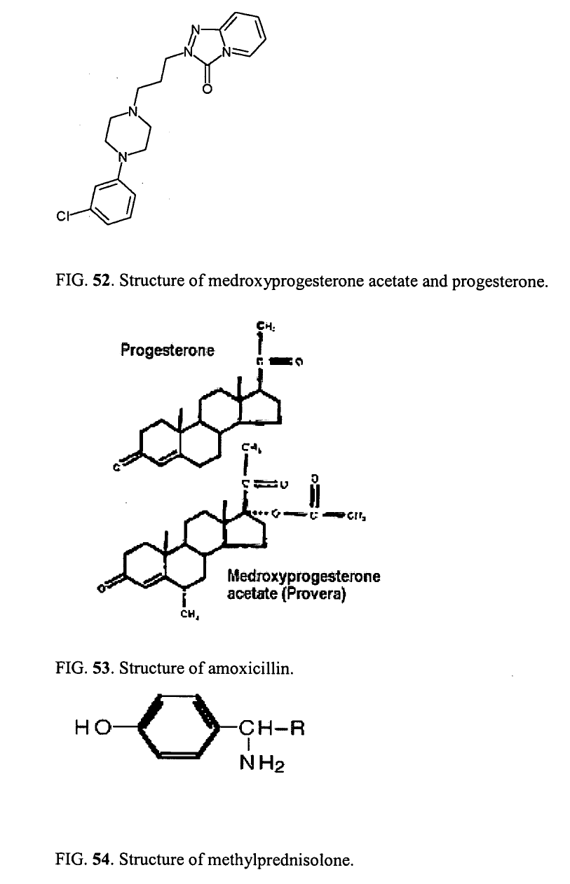

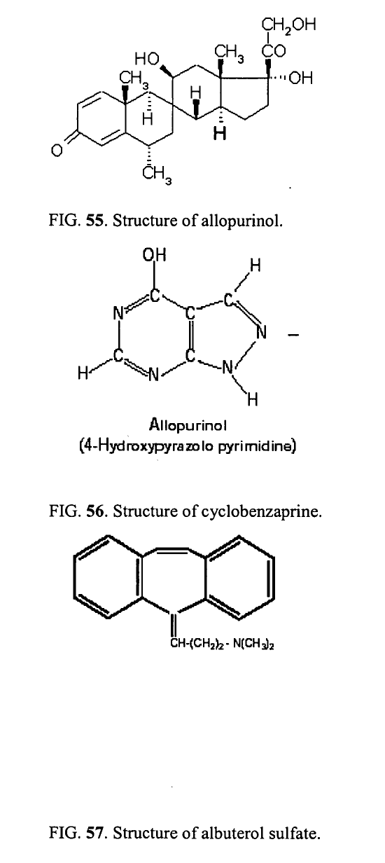

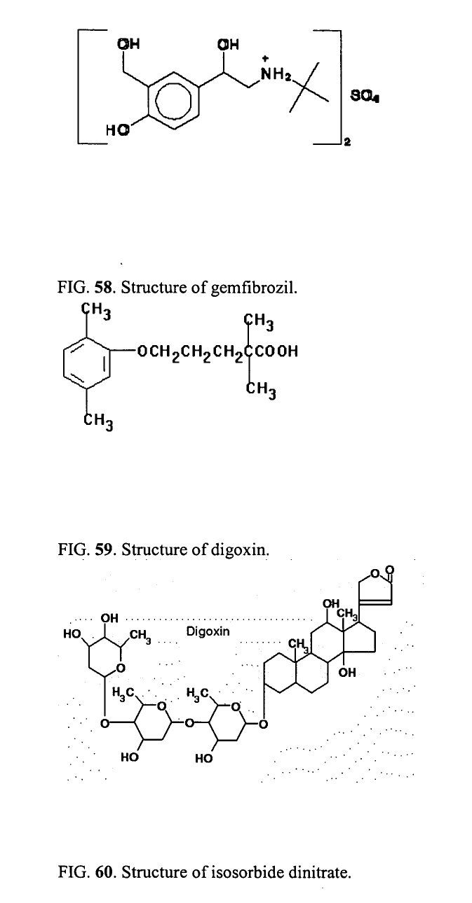

Other agents suitable for ketone-based polymerization include but are not limited to: ciprofloxacin, heparin, liprostin, oxycodone, hydromorphone, Alagebrium (formerly ALT-711), drondarone, eplerenone, albuterol, prednisone, doxycycline, and medroxyprogesterone (refer to FIG. 52).

Activated Aromatic Ring-Based Polymerization

Examples of Polymers of Omeprazole

For each, the monomer can include omeprazole, or any analogue thereof. Omeprazole (refer to FIG. 31) contains an activated benzene ring, which can be used to form polymers as described in examples below.

Polymer with Silico-Omeprazole Side Chains.

The activated aromatic ring of omeprazole is first halogenated with bromine or chlorine in the presence of a catalyst such as FeBr₃ or AlCl₃ using standard methods (refer to FIG. 32). De la Mare, P. B. D. Electrophilic Halogenation. Cambridge University Press: Cambridge, 1976.; Eisch, J. J. Adv. Heterocycl. Chem., 1966, 7, 1. The halogen will preferentially brominate or chlorinate the benzene ring fused to the imidazole at the carbon between the oxygenated and iminated carbons. Any aliphatic polymer with tri-substituted silicon side chains (SiR₃) is reacted with the halogenated omeprazoles using standard methods. The silicon will displace the halogen, forming a silicon bridge between the polymer backbone and the omeprazoles that is degradable under physiologic conditions. Additionally, the polymer backbone can be made of amides, aromatic rings, or other hydrolysable link that can be degraded in vivo either by proteases or by non-enzymatic hydrolysis. In this way, multiple omeprazoles are added to a single polymer backbone. The degree of saturation of omeprazole on the polymer backbone can be controlled by varying the reaction conditions, such as the concentration of omeprazole, the concentration and composition of the polymer backbone, the concentration of the ferric or other catalyst, the duration of the reaction, the temperature of the reaction, and the like, as is well-known to one skilled in the art.

This technique similarly applies to metals and metalloids other than silicon. Other metals that, once controlled under physiological conditions, could act as a bridge between the polymer backbone and omeprazole include, but are not limited to, magnesium (Mg), lithium (Li), alkyl-mercury (Hg-R), sodium (Na), and di-hydroxyboron (B(OH)₂).

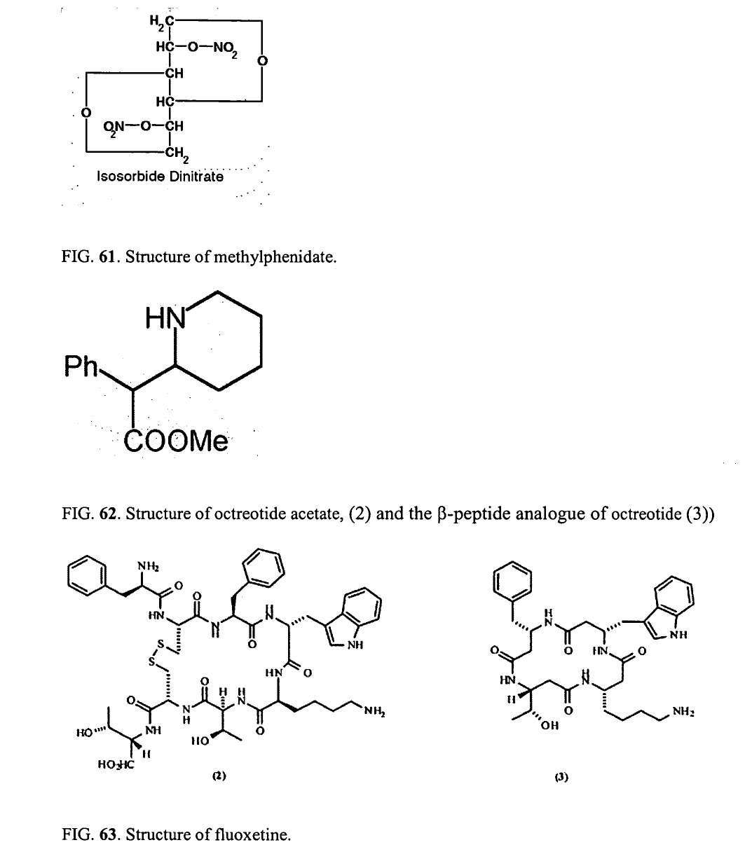

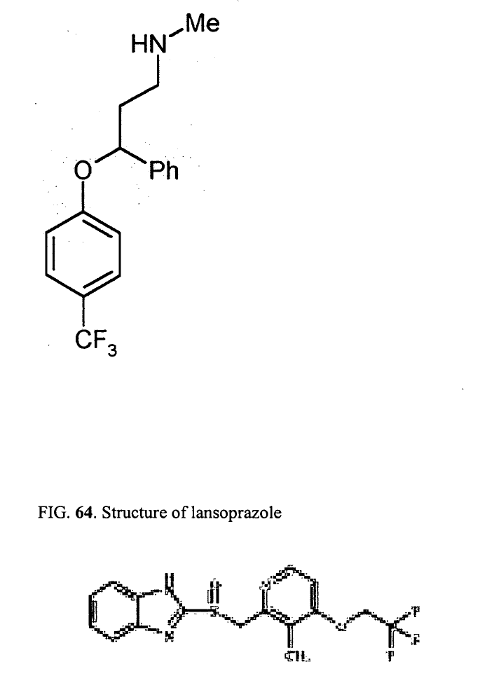

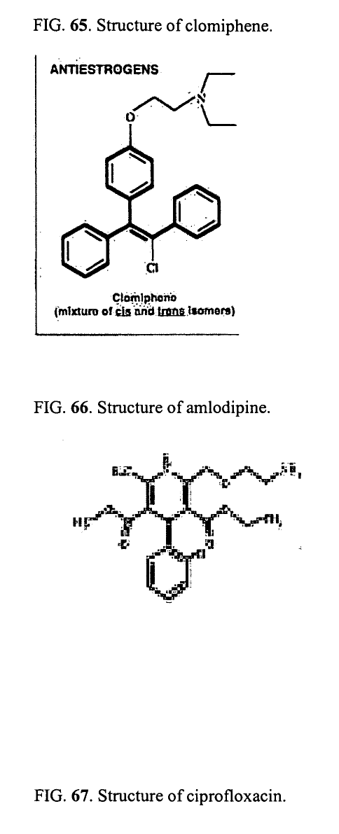

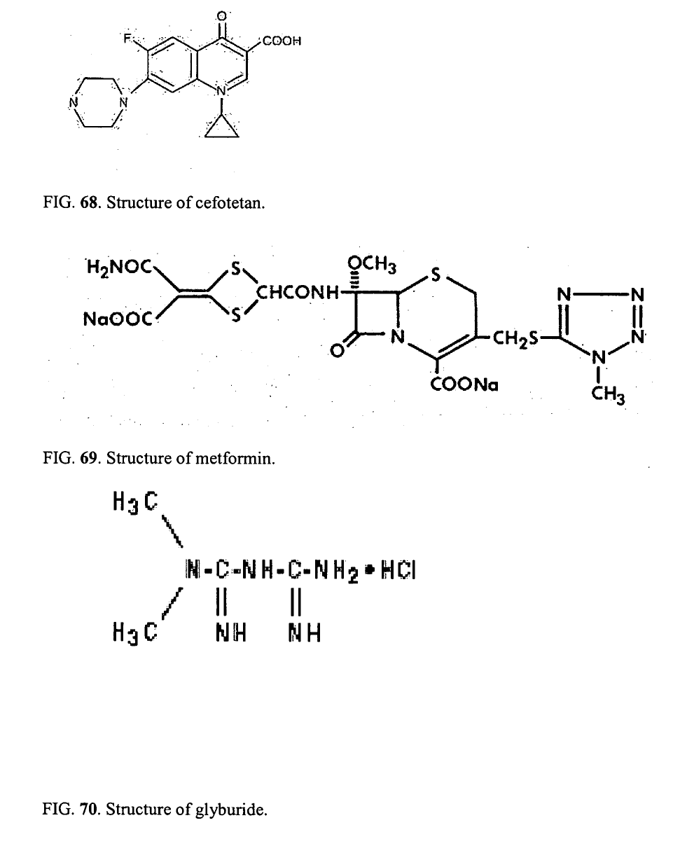

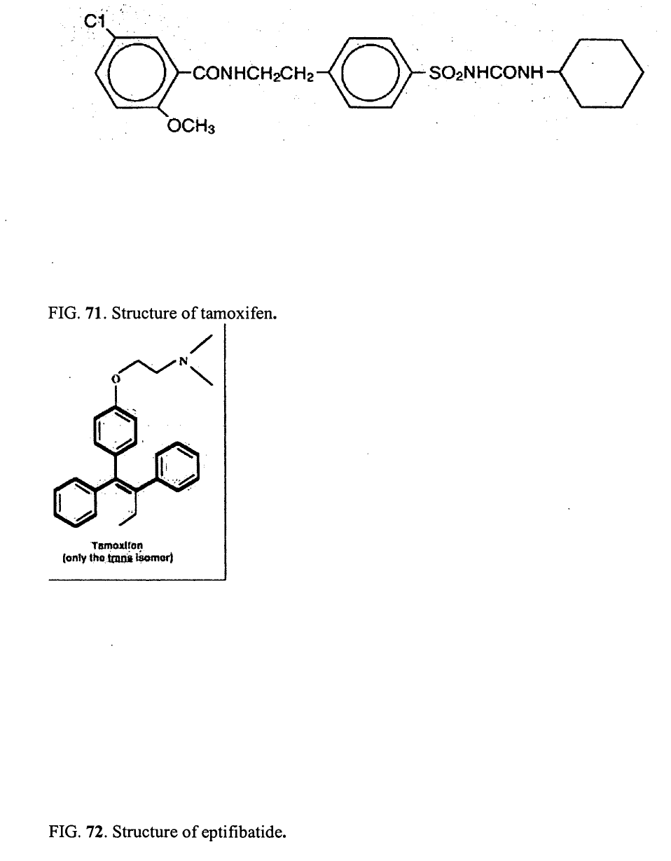

Other agents suitable for activated aromatic ring-based polymerization include but are not limited to: fexofenadine, refecoxib, celecoxib, sildenafil, sertraline, methylphenidate (refer to FIG. 61), metoprolol, octreotide, fluoxetine (refer to FIG. 63), infliximab, lansoprezole (refer to FIG. 64), atorvastatin, clomiphene (refer to FIG. 65), amlodipine (refer to FIG. 66), hydrocodone, trastuzumab, ciprofloxacin (refer to FIG. 67), esomeprazole, cefotetan (refer to FIG. 68), metformin (refer to FIG. 69), glyburide (refer to FIG. 70), tamoxifen (refer to FIG. 71), BMS-232623, neotrophin (AIT-082), c-peptide, cerebrolysin, pentfuside, PRO542, VEGF121, CI-1023, FGF2, neutralase, rNAPc2, natrecor, bivalerudin, tp10, entanercept, tenecteplase, apo a-1 Milano, AGO-1067, rosuvastatin, NK-104, argatroban, abciximab, ibuprofen, naproxen, RSR13, atacand candesartan, valsartan, TBC3711, DAPD, oxycodone, hydromorphone, calanolide a, mycophenylate, tipranavir ranolazine, tracleer bosentan actelion, tezosentan, motefaxin lutetium, capraverine, niaspan, huperzine q, ALT-711, drondarone, melatonin, irbesartan, BMS-207940, phenserine, CP-597,396 (zoniporide, a selective NHAE1 inhibitor), nefiracetam, YM087 (conivaptan), emivirine, liprostin, nifedipine, rofecoxib, xanax® (alprazolam), atenolol, furosemide, triamterene, alprazolam, albuterol (refer to FIG. 57), amoxicillin (refer to FIG. 53), propoxyphene, fluoxetine, verapamil, glyburide, doxazosin, lorazepam, temazepam, amit) riptyline, warfarin, sulfamethoxazole, trimethoprim, diltiazem, clonazepam (refer to FIG. 42), nifedipine, estradiol, doxycycline, diazepam (refer to FIG. 48), clonidine, glipizide (refer to FIG. 50), and trazodone (refer to FIG. 51).

Cyclic Lactam-Based Polymerization

Examples of Polymers of Sildenafil

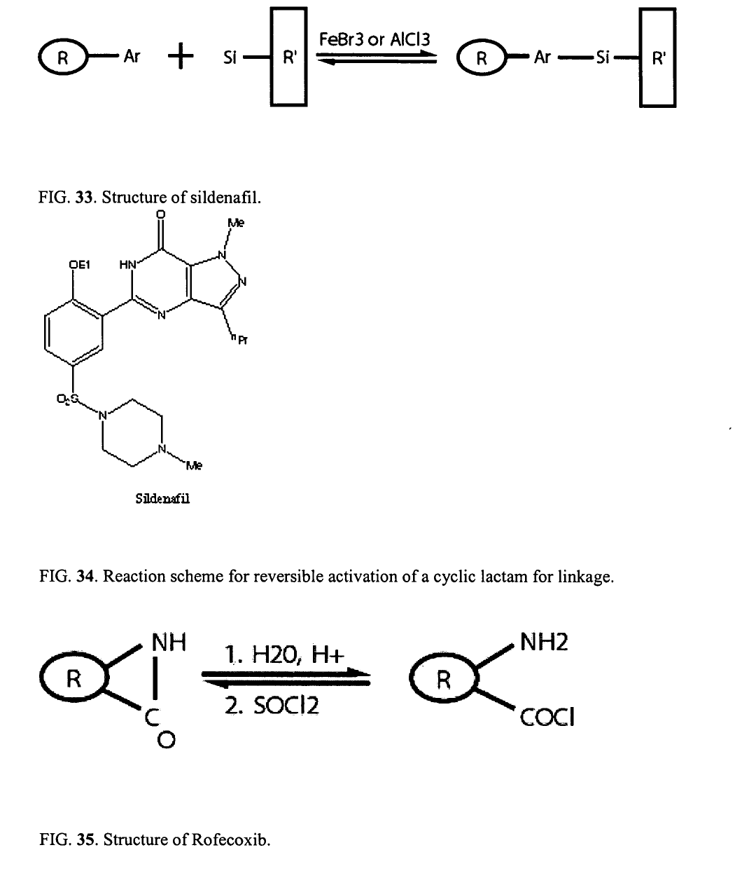

For each, the monomer can include sildenafil (refer to FIG. 33), or any analogue thereof. Sildenafil contains a cyclic lactam that can be degraded to form a 6-amino acid derivative (“sildenafil-δ-derivative”). This degradation can be achieved by using an amidase or through non-enzymatic hydrolysis under acidic conditions. The resulting derivative contains free amine and carboxylic acid termini that can be used to form polymers as described in examples below. After degradation of the polymer, the δ-amino acid readily undergoes condensation to regenerate sildenafil, the original lactam. Bladé-Font, A. Tetrahedron Lett., 1980, 21, 2443.; and Wei, Z.-Y.; Knaus, E. E. Tetrahedron Lett. 1993, 34, 4339. The rate of lactamization can be significantly increased by the presence of enzymes such as pancreatic porcine lipase. Gutman, A. L.; Meyer, E.; Yue, X.; Abell, C. Tetrahedron Lett., 1992, 33, 3943.

Polyaspartate with Sildenafil-δ-Derivative Side Chains.

Please refer to FIG. 34. Sildenafil-δ-derivative can be linked via amide linkages to aspartate or polyaspartate (Aquadew SPA-30, Ajinomoto, Tokyo, Japan). Using EDC activation as described above, the free side chain carboxylate of polyaspartate (or unprotected carboxylate of choice in the case of aspartate), is made amine reactive, then linked to the free amine of sildenafil-δ-derivative. Additionally, the amide linkages of the polyaspartate backbone can be degraded in vivo either by proteases or by non-enzymatic hydrolysis. In this way, multiple sildenafil-δ-derivatives are added to a single backbone of the polyaspartate. The degree of saturation of sildenafil-δ-derivative on the polyaspartate can be controlled by varying the reaction conditions, such as the concentration of sildenafil-δ-derivative, the concentration of sodium polyaspartate, the concentration of catalyst, the duration of the reaction, the temperature of the reaction, and the like, as is well-known to one skilled in the art.

Polyaspartate having sildenafil-δ-derivative amide side chains could also be formed by first forming sildenafil-δ-derivative amides with aspartate monomers. The sildenafil-δ-derivative amide aspartate monomers could then be polymerized by forming amide linkages between the aspartates. The number of sildenafil-δ-derivatives incorporated in each polyaspartate form can be controlled by reacting the sildenafil-δ-derivative derivatized aspartates with native or otherwise derivatized aspartates. Sildenafil-δ-derivative (Sdd)-aspartate (Asp) conjugates may be polymerized in the presence of or absence of native (or other conjugated aspartate). The ratio of native to conjugated aspartate in the polymer will approximate that in the reaction volume, so the degree of sildenafil-δ-derivative saturation in the resulting polymer can be determined in the protocol.

Alternately, sodium polyaspartate (Aquadew SPA-30, Ajinomoto, Tokyo, Japan) is reacted with sildenafil-δ-derivative and any di- or polyol (for example 1,4-butane-diol) in the presence of a sulfuric acid catalyst using standard methods. De Carvalho, M. G. S. et al. Identification of Phosphorylation sites of human 85-kda cytosolic phospholipase A2 expressed in insect cells and present in human monocytes. 1996. J. Biol. Chem 271(12):6987-97. Free carboxylic acid termini on the sodium polyaspartate react with a free hydroxyl and bridege via the other hydroxyl(s) to the free carboxylate of sildenafil-δ-derivative to form an ester linkage which is degradable in aqueous environments under physiologic conditions. Additionally, the amide linkages of the polyaspartate backbone can be degraded in vivo either by proteases or by non-enzymatic hydrolysis. In this way, multiple sildenafil-δ-derivatives are added to a single backbone of the polyaspartate. The degree of saturation of sildenafil-δ-derivative on the polyaspartate can be controlled by varying the reaction conditions, such as the concentration of sildenafil-δ-derivative, the concentration of sodium polyaspartate, the concentration of the sulfuric acid catalyst, the duration of the reaction, the temperature of the reaction, and the like, as is well-known to one skilled in the art. A polyol can be used to form branched polymers or cross-link the polymers as desired.

Polylysine with Mixed Amide-Linked Sildenafil-δ-Derivative.

The free amines of polylysine are reacted with the free amine of sildenafil-δ-derivative using carbonic acid or bicarbonate. Carbonic acid or bicarbonate can be used to form a mixed amide-ester between the amine of sildenafil-δ-derivative and the hydroxyls of PEG or a polyol such as a carbohydrate using methods described in U.S. Pat. No. 6,371,975.

Alternately, polylysine (p-1399, Sigma Chemical Company, St. Louis, Mo.) has free primary amines as termini on each side chain. Using an activating agent such as EDC (Pierce Endogen, Rockford, Ill.), the carboxylate of sildenafil-δ-derivative can be made amine reactive and coupled with the free side chain amine of lysine (then polymerized as before) or of polylysine. The former requires standard protection of the N terminus. The resulting product would have degradable peptide linkages and degradable side chain amides.

Sildenafil-δ-Derivative on Branched Polyethylene Glycol (PEG) Backbone.

Free hydroxyls on a branched polyethylene glycol molecule can be reacted with the free amine on sildenafil-δ-derivative via di- or multi-acids such as citric acid to form esters. Suitable PEG molecules will have three to four branches each and molecular weights below 10,000. Such PEG materials are available from Shearwater Polymers, (Huntsville, Ala., USA), Nippon-Ho (Japan), and Polymer Source (Canada). The resulting mixed ester-amide linkages are degradable in aqueous environments under physiologic conditions.

Alternately, the carboxylate of sildenafil-δ-derivative can be activated under acidic conditions or via chemical activating agent to form esters via free hydroxyls on polyethylene glycol (PEG) or other polyol. The PEG can be linear or branched as desired. Suitable branched PEG molecules will have three to four branches each and molecular weights below 10,000. Such PEG materials are available from Shearwater Polymers, (Huntsville, Ala., USA), Nippon-Ho (Japan), and Polymer Source (Canada). The resulting ester linkages are degradable in aqueous environments under physiologic conditions.

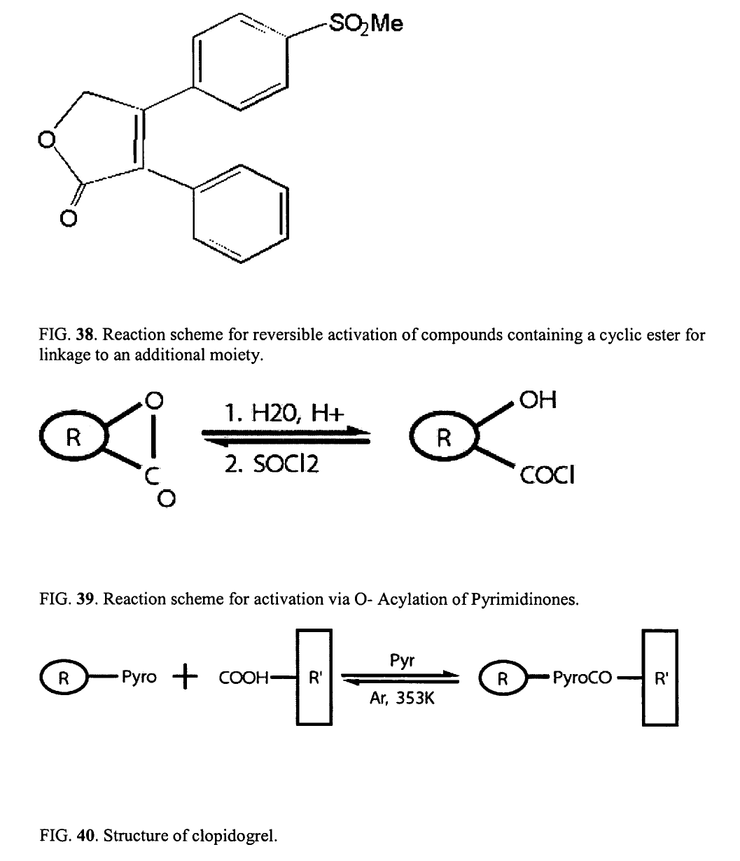

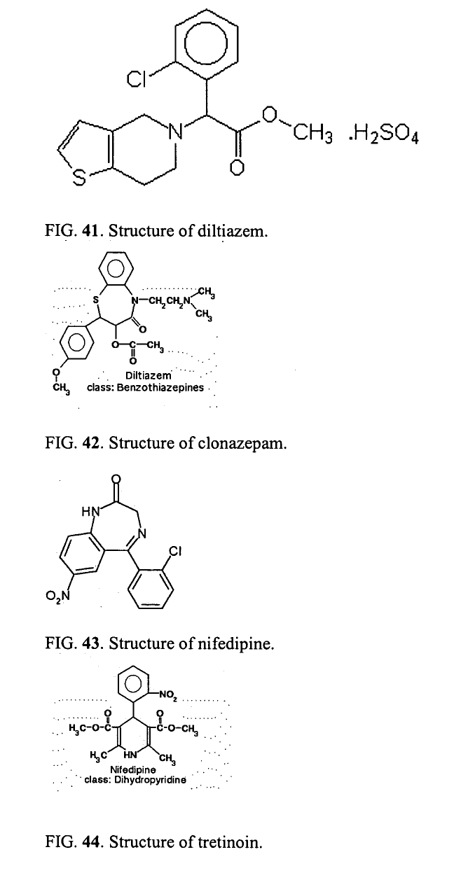

Cyclic Ester-Based Polymerization (Refer to FIG. 38)

Examples of Polymers of Rofecoxib

For each, the monomer can include rofecoxib (refer to FIG. 35), or any analogue thereof. Rofecoxib contains a cyclic ester that can be degraded to form a hydroxyl group and an acyl halide terminus (“rofecoxib-OH-derivative”). This degradation can be achieved by using an esterase or through non-enzymatic hydrolysis under acidic conditions. The resulting derivative contains a free hydroxyl terminus that can be used to form polymers as described in examples below. After degradation of the polymer, the hydroxyl group readily reacts with the γ-acyl halide to form an ester bond and regenerate the original rofecoxib molecule. Bently, T. W.; Llewellyn, G.; McAlister, J. A. J. Org. Chem., 1996, 61, 7927; and Kevill, D. N.; Knauss, D. C. J. chem. Soc., Perkin Trans. 2, 1993, 307; and Fleming, I.; Winter, S. B. D. Tetrahedron Lett., 1993, 34, 7287.

Polyaspartate with Rofecoxib-OH-Derivative Ester Side Chains.

Please refer to FIG. 36 for reaction scheme. Sodium polyaspartate (Aquadew SPA-30, Ajinomoto, Tokyo, Japan) is reacted with rofecoxib-OH-derivative in the presence of a sulfuric acid catalyst using standard methods. De Carvalho, M. G. S. et al. Identification of Phosphorylation sites of human 85-kda cytosolic phospholipase A2 expressed in insect cells and present in human monocytes. 1996. J. Biol. Chem 271(12):6987-97. Free carboxylic acid termini on the sodium polyaspartate react with the free hydroxyl of rofecoxib-OH-derivative to form an ester linkage which is degradable in aqueous environments under physiologic conditions. Additionally, the amide linkages of the polyaspartate backbone can be degraded in vivo either by proteases or by non-enzymatic hydrolysis. In this way, multiple rofecoxib-OH-derivatives are added to a single backbone of the polyaspartate. The degree of saturation of rofecoxib-OH-derivative on the polyaspartate can be controlled by varying the reaction conditions, such as the concentration of rofecoxib-OH-derivative, the concentration of sodium polyaspartate, the concentration of the sulfuric acid catalyst, the duration of the reaction, the temperature of the reaction, and the like, as is well-known to one skilled in the art.

Polyaspartate having rofecoxib-OH-derivative ester side chains could also be formed by first forming rofecoxib-OH-derivative esters with aspartate monomers. The rofecoxib-OH-derivative ester aspartate monomers could then be polymerized by forming amide linkages between the aspartates. The number of rofecoxib-OH-derivatives incorporated in each polyaspartate form can be controlled by reacting the rofecoxib-OH-derivative derivatized aspartates with native or otherwise derivatized aspartates. Rofecoxib-OH-derivative (met)-aspartate (Asp) conjugates may be polymerized in the presence of or absence of native (or other conjugated aspartate). The ratio of native to conjugated aspartate in the polymer will be the same as that in the reaction volume, so the degree of rofecoxib-OH-derivative saturation in the resulting polymer can be determined in the protocol.

Polylysine with Rofecoxib-OH-Derivative Side Chains.

Polylysine (p-1399, Sigma Chemical Company, St. Louis, Mo.) has free primary amines as termini on each side chain. The free primary amines are converted to free thiols using Traut's reagent (Pierce Endogen, Rockford, Ill.) under standard conditions. The reaction can be controlled to convert any number of the side chain amines from a minimum of three to all. The thiol side chains are then covalently bound to the free hydroxyl of rofecoxib-OH-derivative using PMPI (Pierce Endogen), according to the manufacturer's recommendations. PMPI is a heterobifunctional linker which joins free hydroxyls and free thiols.

The PMPI linker could be used with other poly (amino acids) or polypeptides which have free thiols in their side chains.

Polylysine with Amide-Ester Link Rofecoxib-OH-Derivative.

The free amines of polylysine are reacted with the free hydroxyl of rofecoxib-OH-derivative using carbonic acid or bicarbonate. This reaction is described in U.S. Pat. No. 6,371,975 and generates a mixed polymer of rofecoxib-OH-derivative and a free amine-rich peptide with mixed ester-amide linkages. The ester-amide linkages are degradable.

Rofecoxib-OH-Derivative Bound to a Polyethylene Glycol (PEG) Backbone.

As described in U.S. Patent Publication No. U.S. 2002/0055518A1, free thiols can be generated on rofecoxib-OH-derivative. The free thiols on the rofecoxib-OH-derivative may then be reacted with PEG to produce a composition using a linker such as PMPI which joins free hydroxyls and sulfhydryls. Alternately, carbonic acid or bicarbonate can be used to form a mixed ester between the hydroxyls of rofecoxib-OH-derivative and the hydroxyls of PEG using methods described in U.S. Pat. No. 6,371,975.

Rofecoxib-OH-Derivative on Branched Polyethylene Glycol (PEG) Backbone.

Free hydroxyls on a branched polyethylene glycol molecule can be reacted with the free hydroxyl on rofecoxib-OH-derivative, to form esters. Suitable PEG molecules will have three to four branches each and molecular weights below 10,000. Such PEG materials are available from Shearwater Polymers, (Huntsville, Ala., USA), Nippon-Ho (Japan), and Polymer Source (Canada). The resulting mixed diester linkages are degradable in aqueous environments under physiologic conditions.

Polymerized Rofecoxib-OH-Derivative Ascorbic Acid Conjugates.

Rofecoxib-OH-derivative is reacted with ascorbic acid to produce an ester linkage according to well-known techniques; see U.S. Patent Publication Nos. U.S. 2002/0031557 A1; U.S. 2002/0037314 A1; and U.S. 2001/0041193 A1; and Maugard, T., et al. (2000). Studies of vitamin ester synthesis by lipase-catalyzed transesterification in organic media. Biotechnol. Prog. 16(3):358-362. The rofecoxib-OH-derivative ascorbic acid conjugates are then polymerized via free hydroxyls on the ascorbic acid and/or rofecoxib-OH-derivative or anchored to a polymerizable backbone using the techniques described above. Ascorbic acid, also known as vitamin C, is an anti-oxidant which may provide benefits when compositions according to the present invention are used for hyperplasia inhibition or other purposes. Rofecoxib-OH-derivative-ascorbic acid hybrid produced from carbonic acid esterification is shown in FIG. 15. Remaining fee hydroxyls can be derivatized or reacted to add polymerizable groups. Simple rofecoxib-OH-derivative-ascorbic acid hybrid from citric acid esterification is shown in FIG. 16. Free acid groups can react with hydroxyls from adjacent hybrids to cross-link directly or can be reacted with a separate backbone.

Rofecoxib-OH-derivative may be derivatized with other materials which are useful for polymerization and which also provide other functionalities in the polymerized molecules. For example, rofecoxib-OH-derivative may be derivatized with vitamin E, various nitric oxide donors, anti-angiogenic agents, such as angiostatin, HMG-CoA reductase inhibitors, and the like. The resulting heterobifunctional rofecoxib-OH-derivative monomers may then be polymerized to produce compositions according to the present invention using known techniques.

Pyrimidinone-Based Polymerization

Examples of Polymers via O-Acylation of Pyrimidinones

Drugs containing the pyrimidinone ring system may be acylated on the carbonyl (mostly phenolic) oxygen (refer to FIG. 39):

Sildenafil (refer to FIG. 33), as the free base is dissolved in dry pyridine. It is then added to a dry pyridine solution of a mixed anhydride of a carboxylic acid, which can be derived, either from a linker, or from polymer (ex. polyaspartate). The reaction is heated at 80° C. for 2 hrs under argon. At completion, solvent is removed and products are isolated either by silica gel chromatography in the case of the linker adduct or size exclusion in the case of the polyaspartate adduct. Ref: Chem. Pharm. Bull:1988:386.

Thiophene-Based Polymerization

Examples of Polymers of Clopidogrel

For each, the monomer can include clopidogrel, or any analogue thereof. Clopidogrel (refer to FIG. 40) contains a reactive di-substituted benzene ring and a reactive thiophene, both of which can be used to form polymers as described in examples below (following general reaction scheme presented in FIG. 32).

Polymer with Silico-Clopidogrel Side Chains.

Either aromatic ring of clopidogrel is first halogenated with bromine or chlorine in the presence of a catalyst such as FeBr₃ or AlCl₃ using standard methods. De la Mare, P. B. D. Electrophilic Halogenation. Cambridge University Press: Cambridge, 1976.; Eisch, J. J. Adv. Heterocycl. Chem., 1966, 7, 1. The halogen will preferentially brominate or chlorinate the thiophene and will only halogenate the benzene in the presence of excess halogen. Any aliphatic polymer with tri-substituted silicon side chains (SiR₃) is reacted with the halogenated clopidogrels using standard methods. The silicon will displace the halogen, forming a silicon bridge between the polymer backbone and the clopidogrels that is degradable under physiologic conditions. Additionally, the polymer backbone can be made of amides, aromatic rings, or other hydrolysable link that can be degraded in vivo either by proteases or by non-enzymatic hydrolysis. In this way, multiple clopidogrels are added to a single polymer backbone. The degree of saturation of clopidogrel on the polymer backbone can be controlled by varying the reaction conditions, such as the concentration of clopidogrel, the concentration and composition of the polymer backbone, the concentration of the ferric or other catalyst, the duration of the reaction, the temperature of the reaction, and the like, as is well-known to one skilled in the art.

This technique similarly applies to metals and metalloids other than silicon. Other metals that, once controlled under physiological conditions, could act as a bridge between the polymer backbone and clopidogrel include, but are not limited to, magnesium (Mg), lithium (Li), alkyl-mercury (Hg—R), sodium (Na), and di-hydroxyboron (B(OH)₂).

Imidazole-Based Polymerization

Examples of Polymers of Celecoxib

For each, the monomer can include celecoxib (refer to FIG. 25), or any analogue thereof. Celecoxib contains an activated benzene ring and a reactive imidazole, both of which can be used to form polymers as described in examples below (following general reaction scheme presented in FIG. 32).

Polymer with Silico-Celecoxib Side Chains.

An aromatic ring of celecoxib is first halogenated with bromine or chlorine in the presence of a catalyst such as FeBr₃ or AlCl₃ using standard methods; see De la Mare, P. B. D. Electrophilic Halogenation, Cambridge University Press: Cambridge, 1976. Eisch, J. J. Adv. Heterocycl. Chem., 1966, 7, 1. The halogen will preferentially brominate or chlorinate the methylated benzene ring. Any aliphatic polymer with tri-substituted silicon side chains (SiR₃) is reacted with the halogenated celecoxibs using standard methods. The silicon will displace the halogen, forming a silicon bridge between the polymer backbone and the celecoxibs that is degradable under physiologic conditions. Additionally, the polymer backbone can be made of amides, aromatic rings, or other hydrolysable link that can be degraded in vivo either by proteases or by non-enzymatic hydrolysis. In this way, multiple celecoxibs are added to a single polymer backbone. The degree of saturation of celecoxib on the polymer backbone can be controlled by varying the reaction conditions, such as the concentration of celecoxib, the concentration and composition of the polymer backbone, the concentration of the ferric or other catalyst, the duration of the reaction, the temperature of the reaction, and the like, as is well-known to one skilled in the art.

This technique similarly applies to metals and metalloids other than silicon. Other metals that, once controlled under physiological conditions, could act as a bridge between the polymer backbone and celecoxib include, but are not limited to, magnesium (Mg), lithium (Li), alkyl-mercury (Hg—R), sodium (Na), and di-hydroxyboron (B(OH)₂).

Opiates and Anxilytics Polymerizations

Examples of Polymers of Opiates and Anxiolytics

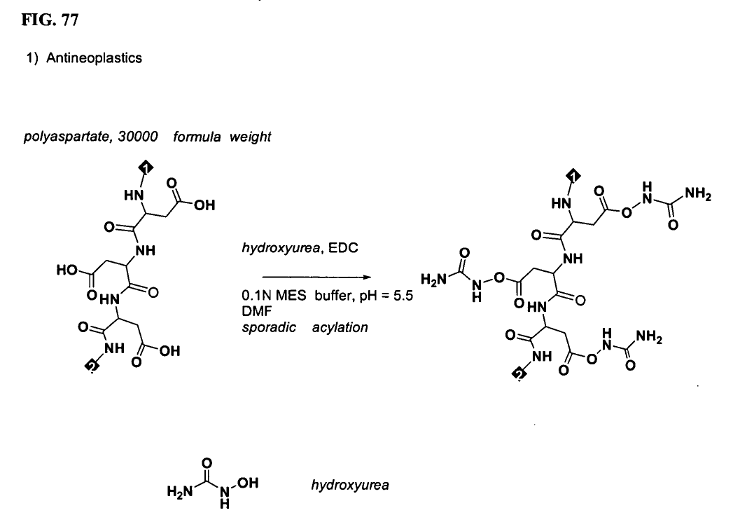

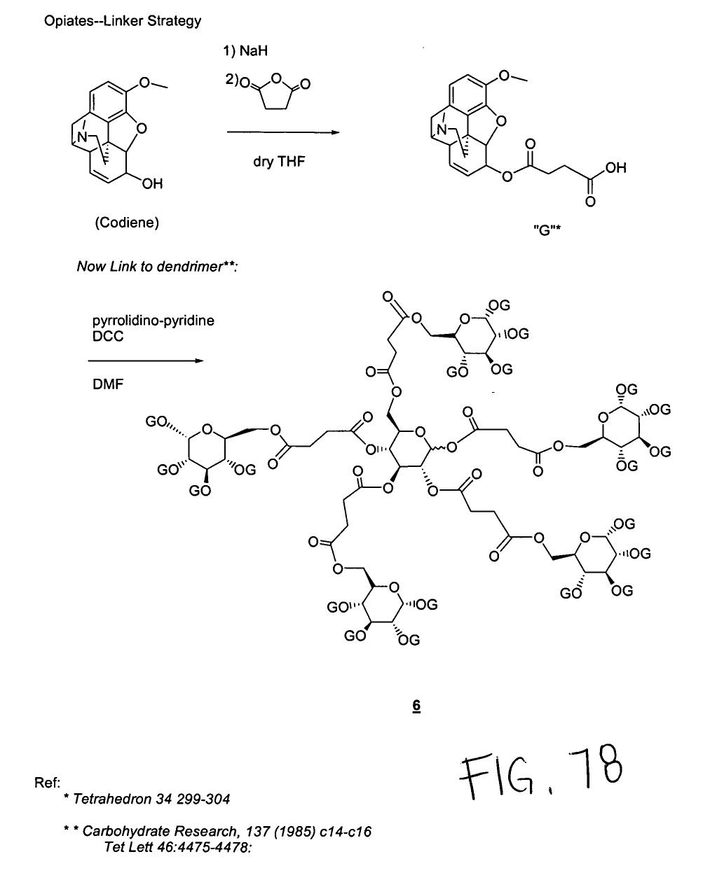

The general procedure is the same as that described in the above examples for linking the polyaspartate backbone molecule to form poly-opiate, e.g., codeine (refer to FIG. 75), a poly-anti-cancer drug, e.g., hydroxyurea (refer to FIG. 77), or polyanxiolytic, e.g., lorazepam (refer to FIG. 76). The material polyaspartate and other reagents are obtained from Pierce Inc.

Instructions for Use: The foregoing therapeutic agent is dissolved in DMSO (dimethylsulfoxide) and added to a solution of polyaspartate in a 0.1M (2-[N-morpholino]ethane sulfonic acid (MES) buffer in a water/DMF solution have a pH of 4.5-5. A coupling agent of 1-ethyl-3-(3-dimethylaminopropyl) carbodiimide hydrochloride (EDC) is added either as a solid or a shortly lived water solution in at least 1:1 equivalence with the agent. The solution is stirred for 2 hrs. at room temperature and then an optional quench with beta-mercaptoethanol or hydroxylamine. Water is added to bring the DMF level to 20% by volume or less for membrane compatability. The reaction is then repeatedly centrifuged and washed with water using a 10K membrane (Pall Corp, Ann Arbor, Mich.) so as to remove materials weighing less than 10000 MW.

An analysis of the resulting drug is carried out by IR, with careful attention for new or added functionality such as esters, amides, amines, and the like. NMR spectroscopy in deuterium oxide can identify added functions such as aryl region protons, and the like.

Linker Strategy for the Opiate (refer to FIG. 78): As outlined in previous examples above, after making a succinate adduct by addition of succinic anhydride to the drug, which has been treated with NaH in dry THF (tetrahydrofolate), it is quenched with saturated NH₄Cl, partitioned with ethyl acetate, dried with brine and MgSO₄, and finally column purified on silica in hexanes/ethyl acetate as a solvent system.

Direct Polymerization

Several compounds have suitable combinations of functionalities to apply direct polymerization using the techniques described above, as will be readily apparent to one skilled in the art. Examples include agents with free amines (primary or secondary), hydroxyls, or unsubstituted sulfonamides and carboxylates, among others. Preferably, each contains 2 or more total of suitable structures. Most preferably, each contains 3 or more suitable functionalities.

Examples of polymers formed by direct polymerization include polymerization of: fexofenadine, infliximab, atorvastatin, trastuzmab, c-peptide, cerebrolysin, pentfuside, PRO542, VEGF121, CI-1023, FGF2, neutralase, rNAPc2, natrecor, bivalarudin, TP-10, entanercept, teneceplase, apo a-1-Milano, AGO-1067, heparin, rosuvastatin, NK-104, liprostin, propoxyphene, eptifibatide, gadopentate, argatroban, abciximab, lisinprol, furosemide, amoxicillin, doxazosin, captopril, albuterol, prednisone, doxycycline (refer to FIG. 47), citicoline, VX-175, cotreotide, hydroxyurea, and emtriciabine.

Phosphate-Based Polymerization

Examples of polymers of other agents suitable for phosphate-based polymerization include but are not limited to: nucleotides and phosphonamides.

Although the invention has been described in reference to its preferred embodiments, those of ordinary skill in the art may make modifications therein without departing from the scope and spirit of the invention which is claimed below. It is expected that certain changes or modifications to the invention disclosed herein may be effected by those skilled in the art without departing from the true spirit and scope thereof as set forth in the claims and the accompanying specification.

1. A polymerized therapeutic agent delivery composition, said composition comprising:
a) at least three agents, each agent containing at least one reactive carboxylate; and
b) a backbone molecule capable of covalently binding to at least one of said agents.

2. The composition of claim 1, wherein said backbone molecule is a polylysine molecule and the reactive carboxylate of at least one of the agent is covalently bound to the polylysine molecule.

3. The composition of claim 1, wherein said backbone molecule is a lysine molecule, the reactive carboxylate of at least one of the agents is covalently bound to the lysine molecule, and the lysine-agent conjugate is polymerized via amide linkages.

4. The composition of claim 1, wherein said backbone molecule is a polyol molecule and the reactive carboxylate of at least one of the agents is covalently bound to the polyol molecule.

5. The composition of claim 4, wherein said polyol molecule is linear or branched.

6. The composition of claim 5, wherein said polyol molecule is glycogen.

7. The composition of claim 1, wherein said backbone molecule is an ascorbic acid molecule and the reactive carboxylate of at least one of the agents is covalently bound to the ascorbic acid to form an ascorbic acid-agent conjugate.

8. The composition of claim 7, wherein said conjugate are polymerized via free hydroxyls on the ascorbic acid.

9. The composition of claim 1, wherein said backbone molecule comprises at least one member selected from the group consisting of vitamin E, nitric oxide donors, anti-angiogenic agents, angiostatin, and HMG-CoA reductase inhibitors.

10. The composition of claim 1, wherein said agent is eptifibatide.

11. The composition of claim 1, wherein said agent comprises at least one member selected from the group consisting of fexofenadine, infliximab, atorvastatin, trastuzumab, cefotetan, gadopentate, lu135252, omapatrilat, neotrophin, c-peptide, cerebrolysin, pentfuside, pro542, VEGF121, CI-1023, FGF2, neutralase, rNAP-c2, natrecor, bivalarudin, TP-10, entanercept, teneceplase, apo a-1-Milano, AGO-1067, heparin, rosuvastatin, NK-104, liprostin, argatroban, abciximab, ibuprofen, naproxen, RSR13, atacand candesartan, valsartan, YM872, lisinopril, furosemid, amoxicillin, captopril, and doxazosin.

12. A polymerized therapeutic agent delivery composition, said composition comprising:
a) at least three agents, each agent containing at least one reactive hydroxyl; and
b) a backbone molecule capable of covalently binding to said agent.

13. The composition of claim 12, wherein said backbone molecule is a polyaspartate molecule and the hydroxyl of at least one of the agents is covalently bound to the polyaspartate molecule.

14. The composition of claim 12, wherein said backbone molecule is an aspartate molecule and the reactive hydroxyl of at least one of the agents is covalently bound to the aspartate molecule and and the aspartate-agent conjugate is polymerized via amide linkages.

15. The composition of claim 12, wherein said backbone molecule is a polyacrylate molecule and the hydroxyl of at least one of the agents is covalently bound to the polyacrylate molecule.

16. The composition of claim 12, wherein said backbone molecule is a polylysine molecule and the reactive hydroxyl of at least one of the agents is covalently bound to the polylysine molecule.

17. The composition of claim 16, wherein said backbone molecule is a polylysine molecule and the polylysine primary amines are converted to sulfhydryls and and the reactive hydroxyl of at least one of the agents is covalently bound to said converted polylysine molecule.

18. The composition of claim 16, wherein said backbone molecule is a polylysine molecule and the polylysine primary amines are converted to thiols and and the reactive hydroxyl of at least one of the agents is covalently bound to said converted polylysine molecule.

19. The composition of claim 12, wherein said backbone molecule is a lysine molecule and the reactive hydroxyl of the agent is covalently bound to the lysine molecule and and the lysine-agent conjugate is polymerized to generate a polymer via ester-amide linkages.

20. The composition of claim 12, wherein said backbone molecule is a polyol molecule and at least one reactive hydroxyl of the agent is converted to a thiol and and the converted agent is covalently bound to the polyol molecule.

21. The composition of claim 20, wherein said polyol molecule is linear or branched.

22. The composition of claim 21, wherein said polyol molecule is glycogen.

23. The composition of claim 12, wherein said backbone molecule is a polyol molecule and the reactive hydroxyl of the agent is covalently bound to the polyol molecule via mixed diester linkages.

24. The composition of claim 12, wherein said backbone molecule is an ascorbic acid molecule and the reactive hydoxyl of the agent is covalently bound to the ascorbic acid to form an ascorbic acid-agent conjugate.

25. The composition of claim 24, wherein said conjugate are polymerized via free hydroxyls on the ascorbic acid.

26. The composition of claim 12, wherein said backbone molecule comprises at least one member selected from the group consisting of vitamin E, nitric oxide donors, anti-angiogenic agents, angiostatin, and HMG-CoA reductase inhibitors.

27. The composition of claim 12, wherein said agent comprises at least one member selected from the group consisting of pravastatin, atorvastatin, fexofenadine, and metroprolol.

28. The composition of claim 12, wherein said agent comprises at least one member selected from the group consisting of octreotide, infliximab, trastuzumab, lu135252, BMS-232623, tecadenoson, c-peptide, cerebrolysin, pentfuside, pro542, VEGF121, C₁₋₁₀₂₃, FGF2, neutralase, rNAP-c2, natrecor, bivalarudin, tp10, entanercept, teneceplase, apo a-1-Milano, AGO-1067, heparin, rosuvastatin, NK-104, liprostin, TBC3711, hydroxyurea, emtricitabine, citicoline, DAPD, carvedilol, oxycodone, hydromorphone, calanolide a, mycophenylate, tipranavir, ranolazine, tracleer bosentan actelion, tezosentan, santidar fondiparinux, pkc inhibitor, angiogenix, motefaxin lutetium, azithromycin, atenolol, albuterol, propoxyphene, prednisone, lorazepam, temaxepam, warfarin, estradiol, doxycycline, codiene, morphine, oxymorphone, and endomorphone.

29. A polymerized therapeutic agent delivery composition, said composition comprising:
a) at least three agents, each agent containing at least one reactive amine; and
a backbone molecule capable of covalently binding to said agent.

30. The composition of claim 29, wherein said backbone molecule is a polyaspartate molecule and the amine of the agent is covalently bound to the polyaspartate molecule.

31. The composition of claim 29, wherein said backbone molecule is an aspartate molecule and the reactive amine of the agent is covalently bound to the aspartate molecule and and the aspartate-agent conjugate is polymerized via amide linkages.

32. The composition of claim 29, wherein said backbone molecule is a polyaspartate molecule and the amine of the agent is converted to an amine-reactive acyl halide and where by the converted agent is covalently bound to the polyaspartate molecule.

33. The composition of claim 29, wherein said backbone molecule is a polylysine molecule and the reactive amine of the agent is covalently bound to the polylysine molecule via amide linkages.

34. The composition of claim 29, wherein said backbone molecule is a polyol molecule and the reactive carboxylate of the agent is covalently bound to the polyol molecule.

35. The composition of claim 34, wherein said polyol molecule is linear or branched.

36. The composition of claim 35, wherein said polyol molecule is glycogen.

37. The composition of claim 29, wherein said backbone molecule is an ascorbic acid molecule and the reactive carboxylate of the agent is covalently bound to the ascorbic acid to form an ascorbic acid-agent conjugate.

38. The composition of claim 37, wherein said conjugate are polymerized via free hydroxyls on the ascorbic acid.

39. The composition of claim 29, wherein said backbone molecule comprises at least one member selected from the group consisting of vitamin E, nitric oxide donors, anti-angiogenic agents, angiostatin, and HMG-CoA reductase inhibitors.

40. The composition of claim 29, wherein said agent is sertraline.

41. The composition of claim 29, wherein said agent comprises at least one member selected from the group consisting of methylphenidate, metroprolol, octreotide, fluoxetine, infliximab, atorvastatin, amlodipine, ciprofloxacin, trastuxumab, esomeprazole, omeprazole, metformin, eptifibatide, gadopentate, neotrophin, c-peptide, cerebrolysin, pentfuside, pro542, VEGF121, CI-1023, FGF2, neutralase, rNAP-c2, natrecor, bivalarudin, TP-10, entanercept, teneceplase, apo a-1-Milano, argatroban, abciximab, lisinopril, hydroxyurea, emtricitabine, citicoline, DAPD, carvedilol, capraverine, cariporide, niaspan, ADA, tmcl25, huperzine q, panzem tmc120, atenolol, furosemide, triamterene, ranitidine, albuterol, amoxicillin, propoxyphene, fluoxetine, doxazosin, sulfamethoxazole, trimetrhoprim, nifedipine, and clonidine.

42. A polymerized therapeutic agent delivery composition, said composition comprising:
a) at least three agents, each agent containing at least one reactive sulfonamide; and
b) a backbone molecule capable of covalently binding to said agent.

43. The composition of claim 42, wherein said agent comprises at least one member selected from the group consisting of sertraline, metformin, rosuvastatin, argatroban, TBC1711, tipranavir, tracleer bosentan actelion, tezosentan, xantidar fondiparinux, cariporide, VX-175, BMS-207940, rofecoxib, furosemide, glyburide, and sulfamethoxazole.

44. A polymerized therapeutic agent delivery composition, said composition comprising:
a) at least three agents, each agent containing at least one reactive ketone; and
b) a backbone molecule capable of covalently binding to said agent.

45. The composition of claim 44, wherein said backbone molecule is a polylysine molecule and the reactive ketone of the agent is covalently bound to the polylysine molecule.

46. The composition of claim 44, wherein said backbone molecule is a lysine molecule and the reactive ketone of the agent is covalently bound to the lysine molecule and and the lysine-agent conjugate is polymerized via amide linkages.

47. The composition of claim 44, wherein said backbone is a carbohydrate and the reactive ketone of the agent is covalently bound to the carbohydrate molecule to produce a hemiketal and ketal linkage.

48. The composition of claim 47, wherein said carbohydrate has at least six hydroxyl groups.

49. The composition of claim 44, wherein said agent is hydrocodone.

50. The composition of claim 44, wherein said agent comprises at least one member selected from the group consisting of ciprofloxacin, heparin, liprostin, oxycodone, hydromorphone, ALT-711, drondarone, eplerenone, albuterol, prednisone, doxycycline, and medroxyprogesterone.

51. A polymerized therapeutic agent delivery composition, said composition comprising:
a) at least three agents, each agent containing at least one activated aromatic ring; and
b) a backbone molecule capable of covalently binding to said agent.

52. The composition of claim 51, wherein said backbone is an aliphatic polymer with metal side chains and the activated aromatic ring of the agent forms a bridge between said backbone and agent.

53. The composition of claim 52, wherein said backbone is an aliphatic polymer with tri-substituted silicon side chain.

54. The composition of claim 52, wherein said backbone is an aliphatic polymer wherein the metal comprises at least one member selected from the group consisting of magnesium, lithium, alkyl-mercury, and di-hydroxyboron.

55. The composition of claim 51, wherein said agent is omeprazole.

56. The composition of claim 51, wherein said agent comprises at least one member selected from the group consisting of fexofenadine, refecoxib, celecoxib, sildenafil, sertraline, methylphenidate, metoprolol, octreotide, fluoxetine, infliximab, lansoprezole, atorvastatin, clomiphene, amlodipine, hydrocodone, trastuzumab, ciprofloxacin, esomeprazole, cefotetan, metformin, glyburide, tamoxifen, BMS-232623 (protease inhibitor), neotrophin (AIT-082), c-peptide, cerebrolysin, pentfuside, pro542, VEGF121, CI-1023, FGF2, neutralase, rNAP-c2, natrecor, bivalerudin, tp10, entanercept, tenecteplase, apo a-1 Milano, AGO-1067, rosuvastatin, NK-104, argatroban, abciximab, ibuprofen, naproxen, RSR13, atacand candesartan, valsartan, TBC3711, DAPD, oxycodone, hydromorphone, calanolide a, mycophenylate, tipranavir ranolazine, tracleer bosentan actelion, tezosentan, motefaxin lutetium, capraverine, niaspan, huperzine q, ALT-711, drondarone, melatonin, irbesartan, BMS-207940, Phenserine, CP-597,396, nefiracetam, YM087, emivirine, liprostin, nifedipine, rofecoxib, xanax, atenolol, furosemide, triamterene, alprazolam, albuterol, amoxicillin, propoxyphene, fluoxetine, verapamil, glyburide, doxazosin, lorazepam, temazepam, amitriptyline, warfarin, sulfamethoxazole, trimethoprim, diltiazem, clonazepam, nifedipine, estradiol, doxycycline, diazepam, clonidine, glipizide, and trazodone.

57. A polymerized therapeutic agent delivery composition, said composition comprising:
a) at least three agents, each agent containing at least one reactive cyclic lactam; and
b) a backbone molecule capable of covalently binding to said agent.

58. The composition of claim 57, wherein said backbone molecule is a polyaspartate molecule and the cyclic lactum of the agent is converted to an amino acid derivative and and the converted agent is covalently bound to the polyaspartate molecule.

59. The composition of claim 57, wherein said backbone molecule is an aspartate molecule and the cyclic lactum of the agent is converted to an amino acid derivative and the converted agent is covalently bound to the aspartate and and the aspartate-agent conjugate can be polymerized via amide linkages.

60. The composition of claim 57, wherein said backbone molecule is a sodium polyaspartate molecule and the cyclic lactum of the agent is converted to an amino acid derivative and the converted agent is covalently bound to the sodium polyaspartate molecule.

61. A polymerized therapeutic agent delivery composition, said composition comprising:
a) at least three agents, each agent containing at least one reactive cyclic ester; and
b) a backbone molecule capable of covalently binding to said agent.

62. The composition of claim 61, wherein said backbone molecule is a polyaspartate molecule and the cyclic ester of the agent is converted to a hydroxyl group and and the converted agent is covalently bound to the polyaspartate molecule.

63. The composition of claim 61, wherein said backbone molecule is an aspartate molecule and the cyclic ester of the agent is converted to a hydroxyl group and the converted agent is covalently bound to the aspartate and and the aspartate-agent conjugate can be polymerized via amide linkages.

64. The composition of claim 61, wherein said backbone molecule is a polylysine molecule and the polylysine primary amines are converted to thiols and and the cyclic ester of the agent is converted to a hydroxyl group and and the converted agent is covalently bound to the converted polylysine molecule.

65. The composition of claim 61, wherein said backbone molecule is a lysine molecule and the lysine primary amine is converted to a thiol and and the cyclic ester of the agent is converted to a hydroxyl group and and the converted agent is covalently bound to the converted lysine molecule and and the converted lysine-converted agent conjugate is polymerized via amide linkages.

66. The composition of claim 61, wherein said backbone molecule is a PEG molecule and the cyclic ester of the agent is converted to a hydroxyl group and and the converted agent is covalently bound to the PEG molecule.

67. The composition of claim 61, wherein said backbone molecule is a PEG molecule and the cyclic ester of the agent is converted to a thiol group and and the converted agent is covalently bound to the PEG molecule.

68. The composition of claim 61, wherein said backbone molecule is an ascorbic acid molecule and the cyclic ester of the agent is converted to a hydroxyl group and is covalently bound to the ascorbic acid to form an ascorbic acid-agent conjugate.

69. The composition of claim 68, wherein said conjugate are polymerized via free hydroxyls on the ascorbic acid.

70. The composition of claim 61, wherein said agent is rofecoxib.

71. The composition of claim 61, wherein said backbone molecule comprises at least one member selected from the group consisting of vitamin E, nitric oxide donors, anti-angiogenic agents, angiostatin, and HMG-CoA reductase inhibitors.

72. A polymerized therapeutic agent delivery composition, said composition comprising:
a) at least three agents, each agent containing at least one pyrimidinone ring system; and
b) a backbone molecule capable of covalently binding to said agent.

73. The composition of claim 72, wherein said backbone molecule is a polyaspartate molecule and the pyrimidinones ring system of the agent is acylated on the carbonyl oxygen and and the converted agent is covalently bound to the polyaspartate molecule.

74. A polymerized therapeutic agent delivery composition, said composition comprising:
a) at least three agents, each agent containing at least one reactive di-substituted benzene ring and a reactive thiopene; and
b) a backbone molecule capable of covalently binding to said agent.

75. The composition of claim 74, wherein said backbone is an aliphatic polymer with metal side chains and the activated aromatic ring of the agent forms a bridge between said backbone and agent.

76. The composition of claim 75, wherein said backbone is an aliphatic polymer with tri-substituted silicon side chain.

77. The composition of claim 75, wherein said backbone is an aliphatic polymer wherein the metal comprises at least one member selected from the group consisting of magnesium, lithium, alkyl-mercury, and di-hydroxyboron.

78. The composition of claim 51, wherein said agent is clopidogrel.

79. A polymerized therapeutic agent delivery composition, said composition comprising:
a) at least three agents, each agent containing at least one reactive benzene ring and a reactive imidazole; and
b) a backbone molecule capable of covalently binding to said agent.

80. The composition of claim 79, wherein said backbone is an aliphatic polymer with metal side chains and the activated aromatic ring of the agent forms a bridge between said backbone and agent.

81. The composition of claim 80, wherein said backbone is an aliphatic polymer with tri-substituted silicon side chain.

82. The composition of claim 80, wherein said backbone is an aliphatic polymer wherein the metal comprises at least one member selected from the group consisting of magnesium, lithium, alkyl-mercury, and di-hydroxyboron.

83. The composition of claim 79, wherein said agent is celecoxib.

84. A polymerized therapeutic agent delivery composition, said composition comprising at least three agents, each agent containing at least two reactive functional groups such it is capable of direct polymerization.

85. The composition of claim 84, wherein said agent comprises at least one member selected from the group consisting of fexofenadine, infliximab, atorvastatin, trastuzmab, c-peptide, cerebrolysin, pentfuside, PRO542, VEGF121, CI-1023, FGF2, neutralase, rNAPc2, natrecor, bivalarudin, TP-10, entanercept, teneceplase, apo a-1-Milano, AGO-1067, heparin, rosuvastatin, NK-104, liprostin, propoxyphene, eptifibatide, gadopentate, argatroban, abciximab, lisinprol, furosemide, amoxicillin, doxazosin, captopril, albuterol, prednisone, doxycycline, citicoline, VX-175, cotreotide, hydroxyurea, and emtriciabine.

86. A method for delivering a physiologically and biologically active agent-containing composition to a patient comprising:
administering said composition in the form of a polymerized composition selected from the group consisting of a biodegradable polymer of at least three of said agents; a non-biodegradable polymer of at least three of said agents; a biodegradable polymer of at least three of said agents having a polymerizable moiety polymerized to at least one of said agents; a non-biodegradable polymer of at least three of said agents having a polymerizable moiety polymerized to at least one of said agents; a biodegradable polymer of at least three of said agents having a backbone molecule covalently bound to at least one of said agents; a non-biodegradable polymer of at least three of said agents having a backbone molecule covalently bound to at least one of said agents; a biodegradable polymer where said agent is covalently bound to at least one of said agents through linking moieties; and a non-biodegradable polymer where said agent is covalently bound to at least one of said agents through linking moieties.

87. The method of claim 86, wherein said biologically active agent contains an active hydroxyl group.

88. The method of claim 87, wherein said biologically active agent is opiate or opioid.

89. The method of claim 88, wherein said opiate is selected from the group consisting of codine, codiene, hydrocodine, oxycodone. morphine, hydromorphine, oxymorphone, and endomorphone.
